(19) United States
(12) Patent Application Publication
(10) Pub. No.: US 2022/0268761 A1
(43) Pub. Date: Aug. 25, 2022

(54) THERAPY MONITORING UNDER TREATMENT WITH AN ANTI-ADRENOMEDULLIN (ADM) BINDER

(71) Applicant: AdrenoMed AG, Hennigsdorf (DE)

(72) Inventors: Joachim STRUCK, Berlin (DE); Andreas BERGMANN, Berlin (DE)

(73) Assignee: AdrenoMed AG, Hennigsdorf (DE)

(21) Appl. No.: 16/756,903

(22) PCT Filed: Oct. 18, 2018
(86) PCT No.: PCT/EP2018/078647

§ 371 Date: Apr. 17, 2020

(30) Foreign Application Priority Data
Oct. 18, 2017 (EP) 17197177.3

(51) Int. Cl. G01N 33/50; C07K 16/22

(52) U.S. Cl. CPC G01N 33/5091; G01N 2333/575; C07K 16/22

(57) ABSTRACT

Subject matter of the present invention is a method for monitoring a therapy in a subject, wherein the subject is under treatment with an anti-Adrenomedullin (ADM) binder selected from the group comprising an antibody, antibody-fragment and/or non-Ig scaffold, comprising determining the level of a fragment of pre-pro-Adrenomedullin selected from the group comprising Midregional Proadrenomedullin (MR-proADM), C-terminal Proadrenomedullin (CT-proADM) and/or Proadrenomedullin N-terminal 20 peptide (PAMP) or fragments thereof in a bodily fluid obtained from said subject; and correlating the level of said fragment of pre-pro-Adrenomedullin with the subject's clinical/medical status of health and/or the risk for an adverse outcome and/or the requirement for adapting therapeutic measures.

### FIELD OF THE INVENTION

Subject matter of the present invention is a method for monitoring a therapy in a subject, wherein the subject is under treatment with an anti-Adrenomedullin (ADM) binder selected from the group comprising an antibody, antibody-fragment and/or non-Ig scaffold, comprising determining the level of a fragment of pre-pro-Adrenomedullin selected from the group comprising Midregional Proadrenomedullin (MR-proADM), C-terminal Proadrenomedullin (CT-proADM) and/or Proadrenomedullin N-terminal 20 peptide (PAMP) or fragments thereof in a bodily fluid obtained from said subject; and correlating the level of said fragment of pre-pro-Adrenomedullin with the subject's clinical/medical status of health and/or the risk for an adverse outcome and/or the requirement for adapting therapeutic measures.

### DESCRIPTION OF THE INVENTION

ADM is a circulating peptide known to regulate vasodilation and vascular integrity. Increased plasma ADM concentrations have been described for several life-threatening conditions, including cardiovascular diseases and septic shock. A method for the detection and quantification of bioactive ADM (bio-ADM) is described by WO2013072509. Therein, monoclonal antibodies against the amidated C-terminus and middle portion of bio-ADM were generated and used for an immunoassay for the quantification of bioactive ADM in plasma.

Moreover, it has been found that the administration of an anti-ADM antibody or anti-ADM antibody fragment or anti-ADM non-Ig scaffold binding to ADM may reduce considerably the risk of mortality in a patient having a severe acute disease or acute condition.

In recent studies, it could be shown that induction of sepsis lead to an increase of plasma bio-ADM in animal experiments. Surprisingly, upon administration of an anti-Adrenomedullin (ADM) antibody, the apparent plasma ADM concentration rose considerably quicker and to a higher level than in the vehicle animals. Considering the vast molar excess of the anti-ADM antibody over the endogenous bio-ADM it has to be assumed, that the measured plasma ADM concentration after administration of the anti-ADM antibody in fact mainly represents bio-ADM complexed with the anti-ADM antibody. However, the disproportional increase of plasma bio-ADM observed after administration of anti-ADM antibody in comparison to vehicle animals was not associated with a worse outcome.

In fact, it has been found that—surprisingly—under treatment with an anti-ADM antibody, measurement of bio-ADM is not suitable to monitor the subject's risk for adverse outcome. In contrast, measurement of fragments derived from the ADM-precursor peptide, to which said anti-ADM antibody does not bind, is suitable for monitoring the stimulation or down-regulation of the ADM system in such conditions, as it is not immediately influenced by administration of the anti-ADM antibody and over time correlates with the clinical outcome of a patient who is under treatment with said anti-ADM antibody.

### BACKGROUND

The peptide adrenomedullin (ADM) was described for the first time in 1993 (Kitamura, K., et al. 1993. “Adrenomedullin: A Novel Hypotensive Peptide Isolated From Human Pheochromocytoma”, Biochemical and Biophysical Research Communications, Vol. 192 (2), pp. 553-560) as a novel hypotensive peptide comprising 52 amino acids, which had been isolated from a human pheochromocytome; SEQ ID No.: 1. In the same year, cDNA coding for a precursor peptide comprising 185 amino acids and the complete amino acid sequence of this precursor peptide were also described. The precursor peptide, which comprises, inter alia, a signal sequence of 21 amino acids at the N-terminus, is referred to as “preproadrenomedullin” (pre-proADM). In the present description, all amino acid positions specified usually relate to the pre-proADM which comprises the 185 amino acids and has the sequence according to SEQ ID No: 2.

The mature adrenomedullin peptide is an amidated peptide (ADM-NH₂), which comprises 52 amino acids (SEQ ID No: 1) and which comprises the amino acids 95 to 146 of pre-proADM, from which it is formed by proteolytic cleavage. Mature ADM, bio-ADM and ADM-NH₂ is used synonymously throughout this application and is a molecule according to SEQ ID No.: 1.

To date, substantially only a few fragments of the peptide fragments formed in the cleavage of the pre-proADM have been more exactly investigated, in particular the physiologically active peptides adrenomedullin (ADM) and “PAMP”, a peptide comprising 20 amino acids (22-41) which follows the 21 amino acids of the signal peptide in pre-proADM. The discovery and characterization of ADM in 1993 triggered intensive research activity, the results of which have been summarized in various review articles, in the context of the present description, reference being made in particular to the articles to be found in an issue of “Peptides” devoted to ADM in particular (Editorial, Takahashi, K 2001. Peptides, Vol. 22: 1691 and Eto, T 2001. Peptides Vol. 22: 1693-1711). A further review is (Hinson, et al. 2000. Endocrine Reviews Vol. 21(2): 138-167).

It has furthermore been found that the concentrations of ADM, which can be measured in the circulation and other biological liquids are, in a number of pathological states, significantly above the concentrations to be found in healthy control persons. Thus, the ADM level in patients with congestive heart failure, myocardial infarction, kidney diseases, hypertensive disorders, diabetes mellitus, in the acute phase of shock and in sepsis and septic shock are significantly increased, although to different extents. The PAMP concentrations are also increased in some of said pathological states, but the plasma levels are lower relative to ADM (Eto T 2001. Peptides, Vol. 22: 1693-1711).

Furthermore, it is known that unusual high concentrations of ADM are to be observed in sepsis or in septic shock (Eto et al. 2001. Peptides 22: 1693-1711; Hirata et al. 1996. Journal of Clinical Endocrinology and Metabolism 81(4): 1449-1453; Ehlenz et al. 1997. Exp Clin Endocrinol Diabetes 105: 156-162; Tomoda et al. 2001. Peptides 22: 1783-1794; Ueda et al. 1999 Am. J. Respir. Crit. Care Med. 160: 132-136; Wang et al. 2001. Peptides 22: 1835-1840). The findings are related to the typical hemodynamic changes which are known as typical phenomena of the course of a disease in patients with sepsis and other severe syndromes, such as, for example, SIRS. Adrenomedullin plays pivotal roles during sepsis development (Wang, Shock 1998, 10(5):383-384; Wang et al. 1998. Archives of surgery 133(12): 1298-1304) and in numerous acute and chronic diseases (Parlapiano et al. 1999. European Review for Medical and Pharmacological Sciences 3:53-61; Hinson et al. 2000 Endocrine Reviews 21(2): 138-167).

In the scientific investigations to date, it has been found, inter alia, that ADM may be regarded as a polyfunctional regulatory peptide. It is released into the circulation partially in an inactive form extended by glycine (Kitamura et al. 1998. Biochem. Biophys. Res. Commun. 244(2): 551-555). There is also a binding protein (Pio et al. 2001. The Journal of Biological Chemistry 276(15): 12292-12300), which is specific for ADM and probably likewise modulates the effect of ADM.

Moreover, it has been found that the above-mentioned further physiologically active peptide PAMP formed from pre-proADM likewise exhibits a hypotensive effect, even if it appears to have an action mechanism differing from that of ADM (Eto et al. 2001. Peptides 22: 1693-1711; Hinson et al. 2000 Endocrine Reviews 21(2):138-167; Kuwasako et al. 1997. FEBS Lett 414(1): 105-110; Kuwasaki et al. 1999. Ann. Clin. Biochem. 36: 622-628; Tsuruda et al. 2001. Life Sci. 69(2): 239-245; Kangawa et al. EP 0 622 458).

Several methods were described to measure circulating levels of ADM: either ADM directly or indirectly by determining a more stable fragment of its cognate precursor peptide. Recently a method was published, describing an assay to measure circulating mature ADM (Marino et al. 2014. Crit Care 18: R34).

Other methods to quantify fragments derived from the ADM precursor have been described, e.g. the measurement of MR-proADM (Morgenthaler et al. 2005. Clin Chem 51(10):1823-9), PAMP (Washimine et al. 1994. Biochem Biophys Res Commun 202(2):1081-7) and CT-proADM (EP 2 111 552). A commercial homogeneous time-resolved fluoroimmunoassay for the measurement of MR-proADM in plasma on a fully automated system is available (BRAHMS MR-proADM KRYPTOR; BRAHMS GmbH, Hennigsdorf, Germany) (Caruhel et al. 2009. Clin Biochem 42(7-8): 725-8). As these peptides are generated in a stoichiometric ratio from the same precursor, their plasma levels are correlated to a certain extent.

Plasma concentrations of ADM are elevated in patients with heart failure and correlate with disease severity (Hirayama et al. 1999. J Endocrinol 160: 297-303; Yu et al. 2001. Heart 86: 155-160). High plasma ADM is an independent negative prognostic indicator in these subjects (Poyner et al. 2002. Pharmacol Rev 54: 233-246).

The role of MR-proADM in heart failure was explored in several studies. In the BACH study (Maisel et al. 2010. J. Am. Coll. Cardiol. 55: 2062-2076), MR-proADM was powerfully prognostic for death at 90 days, adding prognostic value beyond natriuretic peptides. Subsequent data from the PRIDE study (Shah et al. 2012. Eur. Heart J. 33: 2197-2205) solidified a potential prognostic role for MR-proADM; among patients MR-proADM had the best area under the curve (AUC) for mortality at 1 year. Similarly, levels of MR-proADM in patients with chronic heart failure (CHF) were strongly correlated with disease severity and elevated levels of the peptide were strongly associated with an increased risk of death at 12 months of follow-up (van Haehling et al. 2010. European Journal of Heart Failure 12: 484-491; Adlbrecht et al. 2009. European Journal of Heart Failure 11: 361-366).

MR-proADM was investigated during treatment in patients with acute decompensated heart failure (Boyer et al. 2012. Congest Heart Fail 18 (2): 91-97): patients whose MR-proADM levels tended to increase during acute therapy had findings associated with persistent congestion. In the 12-24 hour time-period after therapy, patients with elevations of MR-proADM had increased peripheral edema. Kaiser et al. measured MR-proADM in patients with univentricular hearts (Kaiser et al. 2014. Europ J Heart Failure 16: 1082-1088). Levels in patients with a failed Fontan circuit (exhibiting ascites and peripheral edema) were significantly higher as compared to patients without Fontan failure. Moreover, Eisenhut speculated whether treatments leading to a reduction of adrenomedullin levels can reduce the severity and extent of alveolar edema in pneumonia and septicemia (Eisenhut 2006. Crit Care 10: 418).

The role of MR-proADM in the diagnosis and prognosis of sepsis was investigated in some studies. MR-proADM was described as biomarker for differentiating between septic patients and non-septic patients with SIRS (Christ-Crain et al. 2005. Crit Care 9: R816-824; Angeletti et al. 2013. Clin Chem Lab Med 51: 1059-1067). Moreover, several studies reported MR-proADM as prognostic biomarker in sepsis, severe sepsis and septic shock (Christ-Crain et al. 2005. Crit Care 9: R816-824; Suberviola et al. 2012. Swiss Med Wkly 142: w13542; Guiginant et al. 2009. Intensive Care Med 35: 1859-1867; DE LA Torre-Prados et al. 2016. Minerva Anestesiol 82: 760-766; Andaluz-Ojeda et al. 2015. J Infect 71:136-139).

WO-A1 2004/097423 describes the use of an antibody against adrenomedullin for diagnosis, prognosis, and treatment of cardiovascular disorders. Treatment of diseases by blocking the ADM receptor are also described in the art, (e.g. WO-A1 2006/027147, PCT/EP2005/012844) said diseases may be sepsis, septic shock, cardiovascular diseases, infections, dermatological diseases, endocrinological diseases, metabolic diseases, gastroenterological diseases, cancer, inflammation, hematological diseases, respiratory diseases, muscle skeleton diseases, neurological diseases, urological diseases.

It has been found that the administration of an anti-ADM antibody or anti-ADM antibody fragment or anti-ADM non-Ig scaffold binding to ADM may reduce considerably the risk of mortality in a patient having a severe acute disease or acute condition (WO2013/072510, WO2013072511, WO2013072512, WO2013072513, WO2013072514).

### DETAILED DESCRIPTION OF THE INVENTION

Subject-matter of the present application is a method for monitoring a therapy in a subject, wherein the subject is under treatment with a binder selected from the group comprising an anti-Adrenomedullin (ADM) antibody, antibody-fragment and/or non-Ig scaffold binding to SEQ ID NO. 1 (amino acid 1-52) comprising:
determining the level of a fragment of pre-pro-Adrenomedullin selected from the group comprising Midregional Proadrenomedullin (MR-proADM), C-terminal Proadrenomedullin (CT-proADM) and/or Proadrenomedullin N-terminal 20 peptide (PAMP) or fragments thereof in a bodily fluid obtained from said subject; and
correlating said level of the fragment of pre-pro-Adrenomedullin selected from the group comprising MR-proADM, CT-proADM and/or PAMP with the subject's clinical/medical status of health and/or the risk for an adverse outcome and/or the requirement for adapting therapeutic measures, and
wherein for the determination of the level of said fragments at least one binder binds to a region within the amino acid sequence selected from the group comprising SEQ ID NO. 3, SEQ ID NO. 4 and SEQ ID NO. 5, respectively.

The term “subject” as used herein refers to a living human or non-human organism, preferably herein the subject is a human subject, wherein said subject is suffering from diseases or conditions, e.g. a chronic or acute disease or acute condition. Such a disease may be selected from the group comprising severe infections as e.g. meningitis, Systemic inflammatory Response-Syndrome (SIRS), sepsis; other diseases as diabetes, cancer, acute and chronic vascular diseases as e.g. heart failure, myocardial infarction, stroke, atherosclerosis; shock as e.g. septic shock and organ dysfunction as e.g. kidney dysfunction, liver dysfunction, burnings, surgery, traumata.

As used herein, the term “PAMP” comprises both circulating forms of PAMP, namely a biologically inactive C-terminally Glycine-extended PAMP (PAMP-Gly) and a biologically active C-terminally amidated PAMP (PAMP-amide).

Throughout the specification the “anti-ADM antibodies”, or “anti-ADM antibody fragments” or “anti-ADM non-Ig scaffolds” in accordance with the invention are capable to bind ADM, and thus are directed against ADM, and thus can be referred to as “anti-ADM antibodies”, “anti-ADM antibody fragments”, or “anti-ADM non-Ig scaffolds”.

In accordance with the present invention, the administration of an anti-ADM antibody or anti-ADM antibody fragment binding to ADM or anti-ADM non-Ig scaffold binding to ADM is preferably a systemic application.

In another embodiment of the present application fragments of pre-pro-Adrenomedullin that may be determined in a bodily fluid is/are selected from the group comprising:

(Proadrenomedullin N-20 terminal peptide, PAMP): amino acids 22-41 of
preproADM
SEQ ID No. 3
ARLDVASEF RKKWNKWALS R
(Midregional proAdrenomedullin, MR-proADM): amino acids 45-92 of
preproADM
SEQ ID No. 4
ELRMSS SYPTGLADVK AGPAQTLIRP QDMKGASRSP EDSSPDAARI RV
(C-terminal proAdrenomedullin, CT-proADM): amino acids 148-185 of
preproADM
SEQ ID No. 5
RRR RRSLPEAGPG RTLVSSKPQA HGAPAPPSGS APHFL

In another embodiment of the present application said fragment of pre-proAdrenomedullin having at least 5 amino acids is/are selected from the group comprising MR-proADM (SEQ ID No. 4), CT-proADM (SEQ ID No. 5) and/or PAMP (SEQ ID No. 3).

In one embodiment of the present application the level of the fragments of pre-proADM and/or fragments thereof is determined by using at least one binder, wherein said binder binds to a region comprised within the sequence of MR-proADM (SEQ ID No. 4).

In another embodiment of the present application the level of the fragments of pre-pro-ADM and/or fragments thereof is determined by using at least one binder, wherein said binder binds to a region comprised within the sequence of CT-proADM (SEQ ID No. 5).

In another embodiment of the present application the level of the fragments of pre-proADM and/or fragments thereof is determined by using at least one binder, wherein said binder binds to a region comprised within the sequence of PAMP (SEQ ID No. 3).

Subject matter in a particular embodiment of the present application is a method, wherein said fragment may be selected from MR-proADM according to SEQ ID No.: 4 and/or CT-proADM according to SEQ ID No.: 5 and/or PAMP according to SEQ ID No.: 3.

Another embodiment of the present application relates to a method according to the preceding embodiments, wherein the anti-ADM antibody for the treatment of the subject which binds to the N-terminal part, aa 1-21, of adrenomedullin, is a human CDR-grafted antibody or antibody fragment thereof that binds to ADM, wherein the human CDR-grafted antibody or antibody fragment thereof comprises an antibody heavy chain (H chain) comprising:

SEQ ID NO. 6:
GYTFSRYW
SEQ ID NO. 7:
ILPGSGST
and/or
SEQ ID NO. 8:
TEGYEYDGFDY

and/or further comprises an antibody light chain (L chain) comprising:

SEQ ID NO. 9:
QSIVYSNGNTY
SEQ ID NO. 28: (Not mentioned in
the sequence listing due to the
length of 3 amino acids)
RVS
and/or
SEQ ID NO. 10:
FQGSHIPYT.

In another specific embodiment of the present application the anti-ADM antibody for the treatment of the subject is a human monoclonal antibody that binds to ADM or an antibody fragment thereof wherein the heavy chain comprises at least one CDR selected from the group comprising:

SEQ ID NO. 6:
GYTFSRYW
SEQ ID NO. 7:
ILPGSGST
SEQ ID NO. 8:
TEGYEYDGFDY

and wherein the light chain comprises at least one CDR selected from the group comprising:

SEQ ID No. 9:
QSIVYSNGNTY
SEQ ID NO. 28:
RVS
SEQ ID NO. 10:
FQGSHIPYT.

In another embodiment of the present application, the anti-ADM antibody for the treatment of the subject is a human monoclonal antibody that binds to ADM or an antibody fragment thereof wherein the heavy chain comprises the sequences

SEQ ID NO. 6:
GYTFSRYW
SEQ ID NO. 7:
ILPGSGST
SEQ ID NO. 8:
TEGYEYDGFDY

and wherein the light chain comprises the sequences

SEQ ID NO. 9:
QSIVYSNGNTY
SEQ ID NO. 28:
RVS
SEQ ID NO. 10:
FQGSHIPYT.

Another embodiment of the present application relates to a method of the preceding embodiment, wherein said antibody or fragment for the treatment is a human monoclonal antibody or fragment that binds to ADM or an antibody fragment thereof wherein the heavy chain comprises the sequences

CDR1:
SEQ ID NO. 6:
GYTFSRYW
CDR2:
SEQ ID NO. 7:
ILPGSGST
CDR3:
SEQ ID NO. 8:
TEGYEYDGFDY

and wherein the light chain comprises the sequences

CDR1:
SEQ ID NO. 9:
QSIVYSNGNTY
CDR2:
SEQ ID NO. 28:
RVS
CDR3:
SEQ ID NO. 10:
FQGSHIPYT.

Another embodiment of the present application relates to a method of the preceding embodiment, wherein said antibody or fragment for the treatment comprises the following sequences as a VH region:

SEQ ID NO. 11 (AM-VH-C):
QVQLQQSGAELMKPGASVKISCKATGYTFSRYWIE
WVKQRPGHGLEWIGEILPGSGSTNYNEKFKGKATI
TADTSSNTAYMQLSSLTSEDSAVYYCTEGYEYDGF
DYWGQGTTLTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPK
SEQ ID NO. 12 (AM-VH1):
QVQLVQSGAEVKKPGSSVKVSCKASGYTFSRYWIS
WVRQAPGQGLEWMGRILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPK
SEQ ID NO. 13 (AM-VH2-E40):
QVQLVQSGAEVKKPGSSVKVSCKASGYTFSRYWIE
WVRQAPGQGLEWMGRILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPK
SEQ ID NO. 14 (AM-VH3-T26-E55):
QVQLVQSGAEVKKPGSSVKVSCKATGYTFSRYWIS
WVRQAPGQGLEWMGEILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPK;
or
SEQ ID NO. 15 (AM-VH4-T26-E40-E55):
QVQLVQSGAEVKKPGSSVKVSCKATGYTFSRYWIE
WVRQAPGQGLEWMGEILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPK;

and comprises the following sequence as a VL region:

SEQ ID NO. 16 (AM-VL-C):
DVLLSQTPLSLPVSLGDQATISCRSSQSIVYSNGN
TYLEWYLQKPGQSPKLLIYRVSNRFSGVPDRFSGS
GSGTDFTLKISRVEAEDLGVYYCFQGSHIPYTFGG
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC
SEQ ID NO. 17 (AM-VL1):
DVVMTQSPLSLPVTLGQPASISCRSSQSIVYSNGN
TYLNWFQQRPGQSPRRLIYRVSNRDSGVPDRFSGS
GSGTDFTLKISRVEAEDVGVYYCFQGSHIPYTFGQ
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC
SEQ ID NO. 18 (AM-VL2-E40):
DVVMTQSPLSLPVTLGQPASISCRSSQSIVYSNGN
TYLEWFQQRPGQSPRRLIYRVSNRDSGVPDRFSGS
GSGTDFTLKISRVEAEDVGVYYCFQGSHIPYTFGQ
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC.

Another embodiment of the present application relates to a method of the preceding embodiment, wherein said antibody or fragment for the treatment comprises the following sequence as a heavy chain:

SEQ ID No. 26:
QVQLVQSGAEVKKPGSSVKVSCKASGYTFSRYWIE
WVRQAPGQGLEWIGEILPGSGSTNYNQKFQGRVTI
TADTSTSTAYMELSSLRSEDTAVYYCTEGYEYDGI
FDYWGQGTTVTVSSASTKGPSVFPLAPSSKSTSGG
AALGCLVKDYFPEPVTVSWNSGALTSGVHTFPAVL
QSSGLYSLSSVVTVPSSSLGTQTYICNVNHKPSNT
KVDKKVEPKSCDKTHTCPPCPAPELLGGPSVFLFP
PKPKDTLMISRTPEVTCVVVDVSHEDPEVKFNWYV
DGVEVHNAKTKPREEQYNSTYRVVSVLTVLHQDWL
NGKEYKCKVSNKALPAPIEKTISKAKGQPREPQVY
TLPPSRDELTKNQVSLTCLVKGFYPSDIAVEWESN
GQPENNYKTTPPVLDSDGSFFLYSKLTVDKSRWQQ
GNVFSCSVMHEALHNHYTQKSLSLSPGK

and comprises the following sequence as a light chain:

SEQ ID NO: 27
DVVLTQSPLSLPVTLGQPASISCRSSQSIVYSNGN
TYLEWYLQRPGQSPRLLIYRVSNRFSGVPDRFSGS
GSGTDFTLKISRVEAEDVGVYYCFQGSHIPYTFGG
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC

It shall be understood, that the level of a fragment of pre-pro-Adrenomedullin selected from the group comprising MR-proADM (SEQ ID No. 4), CT-proADM (SEQ ID No. 5) and/or PAMP (SEQ ID No. 3) also encompasses fragments thereof, whereby at least one amino acid is missing and said fragments have a length of at least 5 amino acids, more preferred of at least 10 amino acids, most preferred of at least 15 amino acids.

One embodiment of the present application relates to a method of the preceding embodiment, wherein the level of fragments of pre-proAdrenomedullin (of at least 5 amino acids) is determined by using a binder to said fragments (of at least 5 amino acids).

Another embodiment of the present application relates to a method of the preceding embodiment, wherein the binder is selected from the group comprising an antibody, an antibody fragment or a non-Ig-Scaffold binding to fragments of pre-proAdrenomedullin or fragments thereof (of at least 5 amino acids).

A bodily fluid according to the present application is a blood sample. A blood sample may be selected from the group comprising whole blood, serum and plasma. In one embodiment of the present application, said sample is selected from the group comprising human citrate plasma, heparin plasma and EDTA plasma.

An antibody according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length. Full-length immunoglobulin heavy chains are generally about 50 Kd or 446 amino acid in length. Light chains are encoded by a variable region gene at the NH₂-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al. 1987. Eur. J. Immunol. 17:105; Huston et al. 1988. Proc. Natl. Acad. Sci. U.S.A., 85:5879-5883; Bird et al. 1988. Science 242:423-426; Hood et al. 1984 Immunology, Benjamin, N.Y, 2nd ed.; Hunkapiller and Hood 1986. Nature 323:15-16). An immunoglobulin light or heavy chain variable region includes a framework region interrupted by three hypervariable regions, also called complementarity determining regions (CDR's) (see, Sequences of Proteins of Immunological Interest, E. Kabat et al., U.S. Department of Health and Human Services, 1983). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest Immortalization can be accomplished, for example, by EBV infection or by fusing a human B cell with a myeloma or hybridoma cell to produce a trioma cell. Human antibodies can also be produced by phage display methods (see, e.g., Dower et al., PCT Publication No. WO91/17271; McCafferty et al., PCT Publication No. WO92/001047; and Winter, PCT Publication No. WO92/20791), or selected from a human combinatorial monoclonal antibody library (see the Morphosys website). Human antibodies can also be prepared by using transgenic animals carrying a human immunoglobulin gene (for example, see Lonberg et al., PCT Publication No. WO93/12227; and Kucherlapati, PCT Publication No. WO91/10741).

Thus, the antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines and numerous others.

In addition to antibodies, other biopolymer scaffolds are well known in the art to complex a target molecule and have been used for the generation of highly target specific biopolymers. Examples are aptamers, spiegelmers, anticalins and conotoxins.

In a preferred embodiment the antibody format is selected from the group comprising Fv fragment, scFv fragment, Fab fragment, scFab fragment, (Fab)2 fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is the scFab format.

Non-Ig scaffolds may be protein scaffolds and may be used as antibody mimics as they are capable to bind to ligands or antigenes. Non-Ig scaffolds may be selected from the group comprising tetranectin-based non-Ig scaffolds (e.g. described in US 2010/0028995), fibronectin scaffolds (e.g. described in EP 1266 025; lipocalin-based scaffolds ((e.g. described in WO 2011/154420); ubiquitin scaffolds (e.g. described in WO 2011/073214), transferring scaffolds (e.g. described in US 2004/0023334), protein A scaffolds (e.g. described in EP 2231860), ankyrin repeat based scaffolds (e.g. described in WO 2010/060748), microproteins, preferably microproteins forming a cystine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1941867).

One embodiment of the present application relates to a method of the preceding embodiment, wherein the binder for the treatment of the subject is an anti-ADM antibody or an anti-adrenomedullin antibody fragment or anti-ADM non-Ig protein scaffold, wherein said antibody or fragment or scaffold binds to the N-terminal part, aa 1-21, of adrenomedullin:

SEQ ID No. 19
YRQSMNNFQGLRSFGCRFGTC.

Another embodiment of the present application relates to a method of the preceding embodiment, wherein the binder for the treatment of the subject is an anti-ADM antibody or an anti-adrenomedullin antibody fragment or an anti-ADM non-Ig protein scaffold, wherein said antibody or fragment or scaffold binds to the C-terminal part, aa 42-52-amide, of adrenomedullin:

SEQ ID No. 20
APRSKISPQGY-NH₂.

Another embodiment of the present application relates to a method of the preceding embodiment, wherein the binder for the treatment of the subject is an anti-ADM antibody or an anti-adrenomedullin antibody fragment or an anti-ADM non-Ig protein scaffold, wherein said antibody or fragment scaffold binds to the mid-regional part, aa 21-42, of adrenomedullin:

SEQ ID No. 21
CTVQKLAHQIYQFTDKDKDNVA.

Another embodiment of the present application relates to a method according to the preceding embodiments, wherein the determination of the level of said fragments is performed at least once.

Another embodiment of the present application relates to a method according to the preceding embodiments, wherein the determination of the level of said fragments is performed at least once after beginning the treatment with the anti-ADM antibody or anti-adrenomedullin antibody fragment or anti-ADM non-Ig-protein scaffold.

Another embodiment of the present application relates to a method according to the preceding embodiments, wherein the determination of the level of said fragments is performed more than once in one patient after beginning the treatment with the anti-ADM antibody or anti-adrenomedullin antibody fragment or anti-ADM non-Ig-protein scaffold.

Another embodiment of the present application relates to a method according to the preceding embodiments, wherein the determination of the level of said fragments is performed more than twice in one patient after beginning the treatment with the anti-ADM antibody or anti-adrenomedullin antibody fragment or anti-ADM non-Ig-protein scaffold.

Another embodiment of the present application relates to a method according to the preceding embodiments, wherein the determination of the level of said fragments is performed more than three times after beginning the treatment with the anti-ADM antibody or anti-adrenomedullin antibody fragment or anti-ADM non-Ig-protein scaffold.

Another embodiment of the present application relates to a method according to the preceding embodiments, wherein the adverse outcome is selected from the group comprising worsening clinical condition such as worsening organ function, and mortality.

The term “worsening clinical condition” as used herein relates to a worsening of symptoms (e.g. change of clinical parameters defining the progression of a disease), need for hospitalization or death and may be assessed by a medical score (e.g. Acute Physiology And Chronic Health Evaluation (APACHE, APACHE II)).

The term “worsening organ function” according to the present invention includes worsening of renal function (WRF), worsening of cardiovascular function, worsening of hepatic function and worsening of respiratory function and may be assessed by an increasing sequential organ failure assessment (SOFA) score, multiple organ dysfunction score (MODS) or simplified acute physiology score (SAPS, SAPS II).

Another embodiment of the present application relates to a method according to the preceding embodiments, wherein said subject suffers from sepsis or septic shock. In the following clinical criteria for Systemic inflammatory host response (SIRS) sepsis, septic shock will be defined:

1) Systemic inflammatory host response (SIRS) characterized by at least two of the following symptoms:
patients exhibit hypotension (mean arterial pressure is <65 mm Hg)
elevated serum lactate level being >4 mmol/L
blood glucose>7.7 mmol/L (in absence of diabetes)
central venous pressure is not within the range 8-12 mm Hg
urine output is <0.5 mL×kg⁻¹×hr⁻¹
central venous (superior vena cava) oxygen saturation is <70% or mixed venous<65%
heart rate is >90 beats/min
temperature<36° C. or >38° C.
respiratory rate>20/min white cell count<4 or >12×10⁹/L (leucocytes); >10% immature neutrophils.

2) Sepsis
Following at least two of the symptoms mentioned under 1), and additionally a clinical suspicion of new infection, being it:patients exhibit hypotension (mean arterial pressure is <65 mm Hg)
elevated serum lactate level being >4 mmol/L
blood glucose>7.7 mmol/L (in absence of diabetes)
central venous pressure is not within the range 8-12 mm Hg
urine output is <0.5 mL×kg¹×hr¹
central venous (superior vena cava) oxygen saturation is <70% or mixed venous<65%
heart rate is >90 beats/min
temperature<36° C. or >38° C.
respiratory rate>20/min
white cell count<4 or >12×10⁹/L (leucocytes); >10% immature neutrophils,

and additionally a clinical suspicion of new infection, being:
cough/sputum/chest pain
abdominal pain/distension/diarrhoea
line infection
endocarditis
dysuria
headache with neck stiffness
cellulitis/wound/joint infection
positive microbiology for any infection

3) Severe sepsis

Provided that sepsis is manifested in patient, and additionally a clinical suspicion of any organ dysfunction, being:
blood pressure systolic<90/mean; <65 mmHG
lactate>2 mmol/L
Bilirubine>34 μmol/L
urine output<0.5 mL/kg/h for 2 h
creatinine>177 μmon
platelets<100×10⁹/L
SpO₂>90% unless O₂ given

4) Septic shock

At least one sign of end-organ dysfunction as mentioned under 3) is manifested. Septic shock is indicated, if there is refractory hypotension that does not respond to treatment and intravenous fluid administration alone is insufficient to maintain a patient's blood pressure from becoming hypotensive.

Very recently, the definitions for sepsis and septic shock have been reexamined and updated by the sepsis definitions task force group (Singer et al. 2016. JAMA 315 (8): 801-810), which is incorporated here by reference. Sepsis is defined as life-threatening organ dysfunction caused by a dysregulated host response to infection when the body's response to an infection injures its own tissues and organs. Organ dysfunction can be identified as an acute change in total SOFA score≥2 points consequent to the infection. The baseline SOFA score can be assumed to be zero in patients not known to have preexisting organ dysfunction. A SOFA score≥2 reflects an overall mortality risk of approximately 10% in a general hospital population with suspected infection. Even patients presenting with modest dysfunction can deteriorate further, emphasizing the seriousness of this condition and the need for prompt and appropriate intervention, if not already being instituted.

Septic shock is a subset of sepsis in which underlying circulatory and cellular/metabolic abnormalities are profound enough to substantially increase mortality. Patients with septic shock can be identified with a clinical construct of sepsis with persisting hypotension requiring vasopressors to maintain MAP ≥65 mmHg and having a serum lactate level>2 mmol/L (18 mg/dL) despite adequate volume resuscitation.

TABLE 1
Sequential [Sepsis-Related] Organ Failure Assessment Score

| | Score | | | | |
|---|---|---|---|---|---|
| System | 0 | 1 | 2 | 3 | 4 |

Respiration

| | | | | | | | | |
|---|---|---|---|---|---|---|---|---|
| PaO₂/FIO₂, mm Hg | ≥400 | (53.3) | <400 | (53.3) | <300 | (40) | <200 (26.7) with | <100 (13.3) with |
| (kPa) | | | | | | | respiratory support | respiratory support |

Coagulation

| | | | | | |
|---|---|---|---|---|---|
| Platelets, ×10 [symbol] /μL | ≥150 | <150 | <100 | <50 | <20 |

Liver

| | | | | | | | | |
|---|---|---|---|---|---|---|---|---|
| [symbol] mg/ [symbol] L | <1.2 | (20) | 1.2-1.9 | (20-32) | 2.0-5.9 | (33-101) | 6.0-11.9 (102-204) | >12.0 (204) |
| (μmol/L) | | | | | | | | |

| | | | | | |
|---|---|---|---|---|---|
| Cardiovascular | MAP ≥70 | MAP <70 | [symbol] <5 or [symbol] | [symbol] 5.1-15 | [symbol] >15 or |
| | mm Hg | mm Hg | (any [symbol] ) [symbol] | or [symbol] ≤0.1 or | [symbol] >0.1 or |
| | | | | [symbol] ≤0.1 [symbol] | [symbol] >0.1 [symbol] |

Central nervous system

| | | | | | |
|---|---|---|---|---|---|
| Glasgow Coma Scale | 15 | 13-14 | 10-12 | 6-9 | <6 |
| score^{c} | | | | | |

Renal

| | | | | | | | | |
|---|---|---|---|---|---|---|---|---|
| Creatinine, mg/dL | <1.2 | (110) | 1.2-1.9 | (110-170) | 2.0-3.4 | (171-299) | 3.5-4.9 (300-440) | >5.0 (440) |
| (μmol/L) | | | | | | | | |
| Urine output, mL/d | | | | | | | <500 | <200 |

Abbreviations: FIO₂, fraction of inspired oxygen; MAP, mean arterial pressure: PaO₂, partial pressure of oxygen. [symbol]
Adapted from Vincent et al. [symbol]
^{b}Catecholamine doses are given as μg/kg/min for at least 1 hour.
^{c}Glasgow Coma Scale scores range from 3-15; higher score indicates better neurological function. [symbol]
indicates data missing or illegible when filed

b) Catecholamine doses are given as pg/kg/min for at least 1 hour.

c) Glasgow Coma Scale scores range from 3-15; higher score indicates better neurological function.

APACHE II (“Acute Physiology and Chronic Health Evaluation II”) is a severity-of-disease classification system (Knaus et al. 1985. Crit Care Med 13 (10): 818-29), one of several intensive care unit. It is applied within 24 hours of admission of a patient to an ICU: an integer score from 0 to 71 is computed based on several measurements; higher scores correspond to more severe disease and a higher risk of death.

SAPS II is a severity of disease classification system (Le Gall et al. 1993. JAMA 270:2957-2963). Its name stands for “Simplified Acute Physiology Score”, is one of several intensive care unit (ICU) scoring systems and was designed to measure the severity of disease for patients admitted to ICU.

Another embodiment of the present application relates to a method according to the preceding embodiments, wherein said therapeutic measures are selected from the group comprising fluid resuscitation, vasopressors/inotropes, renal replacement therapy, antibiotics, hydrocortisone, insulin, enteral nutrition/parenteral nutrition.

Another embodiment of the present application relates to a method according to the preceding embodiments, wherein said anti-ADM antibody or antibody fragment or non-Ig scaffold does not bind to the fragment of pre-pro-Adrenomedullin selected from the group comprising MR-proADM, CT-proADM and/or PAMP.

Another embodiment of the present application relates to a method according to the preceding embodiments, wherein said fragment of pre-pro-Adrenomedullin selected from the group comprising MR-pro ADM, CT-proADM and/or PAMP is at least 5 amino acids in length, respectively.

Another embodiment of the present application relates to a method according to the preceding embodiments, wherein the level of said fragment of pre-pro-Adrenomedullin selected from the group comprising MR-proADM, CT-proADM and/or PAMP or fragments thereof of at least 5 amino acids is determined by an immunoassay using at least one binder selected from the group comprising a binder to MR-proADM or a fragment thereof and/or to CT-proADM or a fragment thereof and/or to PAMP or a fragment thereof, respectively.

In a specific embodiment of the application an immunoassay is used for determining the level of MR-proADM and/or fragments thereof (having at least 5 amino acids), wherein such immunoassay is a sandwich assay, preferably a fully automated assay.

In a specific embodiment of the application an immunoassay is used for determining the level of CT-proADM and/or fragments thereof (having at least 5 amino acids), wherein such immunoassay is a sandwich assay, preferably a fully automated assay.

In a specific embodiment of the application an immunoassay is used for determining the level of PAMP and/or fragments thereof (having at least 5 amino acids), wherein such immunoassay is a sandwich assay, preferably a fully automated assay.

In one embodiment of the application the immunoassay, which is used for determining the level of MR-proADM and/or CT-proADM and/or PAMP, respectively, may be a so-called POC-test (point-of-care) that is a test technology which allows performing the test within less than 1 hour near the patient without the requirement of a fully automated assay system. One example for this technology is the immunochromatographic test technology.

In one embodiment of the application such an immunoassay is a sandwich immunoassay using any kind of detection technology including but not restricted to enzyme label, chemiluminescence label, electrochemiluminescence label, preferably a fully automated assay.

In one embodiment of the invention such an immunoassay is an enzyme labeled sandwich assay. Examples of automated or fully automated assay comprise assays that may be used for one of the following systems: Roche Elecsys®, Abbott Architect®, Siemens Centauer®, Brahms Kryptor®, BiomerieuxVidas®, Alere Triage®.

A variety of immunoassays are known and may be used for the assays and methods of the present invention, these include: radioimmunoassays (“RIA”), homogeneous enzyme-multiplied immunoassays (“EMIT”), enzyme linked immunoadsorbent assays (“ELISA”), apoenzyme reactivation immunoassay (“ARIS”), dipstick immunoassays and immuno-chromotography assays.

In a specific embodiment of the invention at least one of said two binders is labeled in order to be detected.

The preferred detection methods comprise immunoassays in various formats such as for instance radioimmunoassay (RIA), chemiluminescence- and fluorescence-immunoassays, Enzyme-linked immunoassays (ELISA), Luminex-based bead arrays, protein microarray assays, and rapid test formats such as for instance immunochromatographic strip tests.

In a preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5-or 6-carboxyfluorescein), VIC, NED, Fluorescein, Fluoresceinisothiocyanate (FITC), IRD-700/800, Cyanine dyes, auch as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer, Encyclopedia of chemical technology, 4th ed., executive editor, J. I. Kroschwitz; editor, M. Howe-Grant, John Wiley & Sons, 1993, vol. 15, p. 518-562, incorporated herein by reference, including citations on pages 551-562). Preferred chemiluminescent dyes are acridiniumesters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In the context of the present invention, “binder molecules” are molecules, which may be used to bind target molecules or molecules of interest, i.e. analytes from a sample. Binder molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, binder molecules may for instance be selected from the group comprising a nucleic acid molecule, a carbohydrate molecule, a PNA molecule, a protein, an antibody, a peptide or a glycoprotein. Preferably, the binder molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof.

Chemiluminescent label may be acridinium ester label, steroid labels involving isoluminol labels and the like.

Enzyme labels may be lactate dehydrogenase (LDH), creatine kinase (CPK), alkaline phosphatase, aspartate aminotransferase (AST), alanine aminotransferase (ALT), acid phosphatase, glucose-6-phosphate dehydrogenase and so on.

In a specific embodiment of the invention the threshold is within a threshold range for plasma MR-proADM that is between 0.5 and 1.5 nmol/L, preferably between 0.7 and 1 nmol/L, most preferred a threshold of 0.8 nmol/L is applied.

In a specific embodiment of the invention the threshold is within a threshold range for plasma CT-proADM that is between 85 and 350 pmol/L, preferably between 100 and 250 pmol/L, most preferred a threshold of 150 pmol/L is applied.

In a specific embodiment of the invention a threshold for plasma PAMP-amide that is between 0.3 and 1.2 pmol/L, preferably between 0.4 and 1.0 pmol/L, most preferred a threshold of 0.8 pmol/L is applied.

In a specific embodiment of the invention a threshold for plasma PAMP-glycine that is between 0.5 and 2.0 pmol/L, preferably between 0.7 and 1.8 pmol/L, most preferred a threshold of 1.5 pmol/L is applied.

Another embodiment of the present application relates to a method according to the preceding embodiments, wherein the binder is selected from the group comprising an antibody, an antibody fragment or a non-Ig Scaffold binding to MR-proADM or a fragment thereof and/or to CT-proADM or a fragment thereof and/or PAMP or a fragment thereof, respectively.

Another embodiment of the present application relates to a method according to the preceding embodiments, wherein said Anti-Adrenomedullin (ADM) antibody or an anti-ADM antibody fragment binding to adrenomedullin or anti-ADM non-Ig-protein scaffold binding to adrenomedullin is monospecific.

Another embodiment of the present application relates to a method according to the preceding embodiments, wherein said antibody or fragment or scaffold exhibits a binding affinity to ADM of at least 10⁻⁷ M.

Another embodiment of the present application relates to a method according to the preceding embodiments, wherein said Anti-Adrenomedullin (ADM) antibody or anti-ADM antibody fragment or anti-ADM non-Ig-protein scaffold binding to adrenomedullin exhibits a binding affinity to ADM of at least 10⁻⁷ M wherein said binding affinity is determined by label-free surface plasmon resonance using a Biacore 2000 system.

Another embodiment of the present application relates to a method according to the preceding embodiments, wherein for the correlation an elevated level of said fragment of pre-pro-Adrenomedullin selected from the group comprising MR-proADM, CT-proADM and/or PAMP or fragments thereof above a certain threshold is predictive for an enhanced risk for an adverse outcome, and/or a level of said fragment of pre-pro-Adrenomedullin or fragments thereof below a certain threshold is predictive for a reduced risk for an adverse outcome.

As used herein, the term “elevated level” means a level above a certain threshold level.

Another embodiment of the present application relates to a method according to the preceding embodiments, wherein said threshold is an upper concentration determined for a healthy reference population, such as the 90th, 95th or 99th percentile.

Another embodiment of the present application relates to a method according to the preceding embodiments, wherein for the correlation of the level of the fragment of pre-pro-Adrenomedullin the determination of the level is performed at least twice and wherein a decrease of the second measured level of said fragment in comparison to the measured first level of said fragment is predictive for a reduced risk for an adverse outcome.

Another embodiment of the present application relates to a method according to the preceding embodiments, wherein for the correlation of the level of the fragment of pre-pro-Adrenomedullin the determination of the level is performed at least twice and wherein an increase of the second measured level of said fragment in comparison to the measured first level of said fragment is predictive for an enhanced risk for an adverse outcome.

The term “risk”, as used herein, relates to the probability of suffering from an undesirable event or effect (e.g. a disease).

Another embodiment of the present application relates to a method according to the preceding embodiments, wherein a decrease of the level of the fragment of pre-pro-Adrenomedullin selected from the group comprising MR-proADM, CT-proADM and/or PAMP or fragments thereof, is predictive for a reduced risk for an adverse outcome.

Another embodiment of the present application relates to a method according to the preceding embodiments, wherein an increase of the level of the fragment of pre-pro-Adrenomedullin selected from the group comprising MR-proADM, CT-proADM and/or PAMP or fragments thereof, is predictive for an enhanced risk for an adverse outcome.

Another embodiment of the present application relates to a method according to the preceding embodiments, wherein said decrease is characterized by an improvement of the subject's clinical/medical status of health and/or halving the concentration of said fragment of pre-pro-Adrenomedullin.

Another embodiment of the present application relates to a method according to the preceding embodiments, wherein said increase is characterized by a worsening of the subject's clinical/medical status of health and/or doubling the concentration of said fragment of pre-pro-Adrenomedullin.

Another embodiment of the present application relates to a method according to the preceding embodiments, wherein an assay is used for determining the level of MR-pro ADM, wherein the assay sensitivity of said assay is able to quantify MR-proADM of healthy subjects and is <0.5 nmol/L, preferably <0.4 nmol/L and more preferably <0.2 nmol/L.

Another embodiment of the present application relates to a method according to the preceding embodiments, wherein an assay is used for determining the level of CT-proADM, wherein the assay sensitivity of said assay is able to quantify CT-proADM of healthy subjects and is <100 pmol/L, preferably <75 pmol/L and more preferably <50 pmol/L.

Another embodiment of the present application relates to a method according to the preceding embodiments, wherein an assay is used for determining the level of PAMP-amide, wherein the assay sensitivity of said assay is able to quantify PAMP-amide of healthy subjects and is <0.3 pmol/L, preferably <0.2 pmol/L and more preferably <0.1 pmol/L.

Another embodiment of the present application relates to a method according to the preceding embodiments, wherein an assay is used for determining the level of PAMP-glycine, wherein the assay sensitivity of said assay is able to quantify PAMP-glycine of healthy subjects and is <0.5 pmol/L, preferably <0.25 pmol/L and more preferably <0.1 pmol/L.

Another embodiment of the present application relates to a method according to the preceding embodiments, wherein an assay is used for determining the level of MR-proADM and/or CT-proADM and/or PAMP, and wherein the assay sensitivity is <0.5 nmol/L, preferably <0.4 nmol/L and more preferably <0.2 nmol/L for MR-pro ADM and/or <100 pmol/L, preferably <75 pmol/L and more preferably <50 pmol/L for CT-proADM, and/or <0.3 pmol/L, preferably <0.2 pmol/L and more preferably <0.1 pmol/L for PAMP.

The proADM levels or fragments thereof, of the present invention have been determined with the described assays, as outlined in the examples. The mentioned threshold values above might be different in other assays, if these have been calibrated differently from the assay systems used in the present invention. Therefore, the mentioned cut-off values above shall apply for such differently calibrated assays accordingly, taking into account the differences in calibration. One possibility of quantifying the difference in calibration is a method comparison analysis (correlation) of the assay in question with the respective biomarker assay used in the present invention by measuring the respective biomarker (e.g. bio-ADM) in samples using both methods. Another possibility is to determine with the assay in question, given this test has sufficient analytical sensitivity, the median biomarker level of a representative normal population, compare results with the median biomarker levels as described in the literature and recalculate the calibration based on the difference obtained by this comparison. With the calibration used in the present invention, samples from normal (healthy) subjects have been measured: median plasma bio-ADM (mature ADM-NH₂) was 24.7 pg/ml, the lowest value 11 pg/ml and the 99^{th} percentile 43 pg/ml. Alternatively, commercially available control samples could be used for adjustment of different calibrations (e.g. ICI Diagnostics, Berlin, Germany).

The plasma median MR-proADM concentration in normal (healthy) subjects was 0.41 (interquartile range 0.23-0.64) nmol/L (Smith et al. 2009. Clin Chem 55:1593-1595) using the automated sandwich fluorescence assay for the detection of MR-proADM as described in Caruhel et al. (Caruhel et al. 2009. Clin Biochem 42:725-8).

The plasma median concentration of CT-proADM in normal healthy subjects (n=200) was 77.6 pmol/L (min 46.6 pmol/L, max 136.2 pmol/L) and the 95% percentile was 113.8 pmol/L (EP 2 111 552 B1).

The plasma concentration of PAMP-amide in normal healthy subjects (n=51) was 0.51±0.19 pmol/L (mean±SD) (Hashida et al. 2004. Clin Biochem 37: 14-21).

The plasma concentration of PAMP-glycine in normal healthy subjects (n=51) was 1.15±0.38 pmol/L (mean±SD) (Hashida et al. 2004. Clin Biochem 37: 14-21).

Another embodiment of the present application relates to a method according to the preceding embodiments, wherein said bodily fluid may be selected from the group comprising blood, serum, plasma, urine, cerebrospinal fluid (CSF), and saliva.

Another embodiment of the present application relates to a method according to the preceding embodiments, wherein additionally at least one clinical parameter is determined and additionally considered in the correlations selected from the group comprising age, gender, SOFA score (or subscores thereof), SAPSII score, BUN, Sodium, Potassium, Creatinine, Bilirubin, Platelets count, arterial pH, Hematocrit, White Blood count, HCO³⁻, invasive mechanical ventilation/non-invasive mechanical ventilation, hemodynamic characteristics (incl. Blood pressure, systolic and diastolic, Mean arterial pressure, Central venous pressure, Heart rate), Fluid balance, Urine output, Base excess, Chloride, CRP, PCT, BNP or NT-proBNP, Troponin T or Troponin I, Pro-Enkephalin, Hemoglobin, Glucose, Lactate, INR, Alkaline phosphatase, AST, ALT, Gamma GT, Total protein, Albumin, Temperature, Respiratory rate, PaO₂ and FiO₂, therapeutic measures (fluid resuscitation, vasopressors/inotropes, Renal replacement therapy, antibiotics, Hydrocortisone, Insulin, Enteral nutrition/Parenteral nutrition, pre-existing co-morbidities, chronic medication.

Another embodiment of the present application relates to a method according to the preceding embodiments in order to stratify said subjects into risk groups.

Another embodiment of the present application relates to a method according to the preceding embodiments, wherein said subject is stratified into groups of patients wherein one group comprises patients in need of therapy and the other group comprises patient that are not in need of therapy.

Further embodiments within the scope of the present invention are set out below:
1. A method for monitoring a therapy in a subject, wherein the subject is under treatment with a binder selected from the group comprising an anti-Adrenomedullin (ADM) antibody, anti-body fragment and/or non-Ig Scaffold binding to SEQ ID NO. 1 (amino acid 1-52), comprising
   determining the level of a fragment of pre-pro-Adrenomedullin selected from the group comprising Midregional Proadrenomedullin (MR-proADM), C-terminal Proadrenomedullin (CT-proADM) and/or Proadrenomedullin N-terminal 20 peptide (PAMP) or fragments thereof in a bodily fluid obtained from said subject; and
   correlating said level of the fragment of pre-pro-Adrenomedullin selected from the group comprising MR-proADM, CT-proADM and/or PAMP with the subject's clinical/medical status of health and/or the risk for an adverse outcome and/or the requirement for adapting therapeutic measures, and
   wherein for the determination of the level of said fragments at least one binder binds to a region within the amino acid sequence selected from the group comprising SEQ ID NO. 3, SEQ ID NO. 4 and SEQ ID NO. 5, respectively.
2. A method according to embodiment 1, wherein the binder for the treatment is an anti-ADM antibody or an anti-adrenomedullin antibody fragment or an anti-ADM non-Ig protein scaffold, wherein said antibody or fragment or scaffold binds to the N-terminal part, aa 1-21, of adrenomedullin:
   YRQSMNNFQGLRSFGCRFGTC; SEQ ID No. 19.
3. A method according to embodiment 1 to 2, wherein the binder for the treatment is an anti-ADM antibody or an anti-adrenomedullin antibody fragment or an anti-ADM non-Ig protein scaffold, wherein said antibody or fragment or scaffold binds to the C-terminal part, aa 42-52-amide, of adrenomedullin:

SEQ ID No. 20
APRSKISPQGY-NH₂.

4. A method according to embodiment 1 to 3, wherein the binder for the treatment is an anti-ADM antibody or an anti-adrenomedullin antibody fragment or an anti-ADM non-Ig protein scaffold, wherein said antibody or fragment or scaffold binds to the mid-regional part, aa 21-42, of adrenomedullin:

SEQ ID No. 21
CTVQKLAHQIYQFTDKDKDNVA.

5. A method according to any of the preceding embodiments, wherein the determination of the level of said fragments is performed at least once.
6. A method according to any of the preceding embodiments, wherein the adverse outcome is selected from the group comprising worsening clinical condition such as worsening organ function, and mortality.
7. A method according to any of the preceding embodiments, wherein the worsening of the clinical condition relates to a worsening of symptoms (e.g. change of clinical parameters defining the progression of a disease), need for hospitalization or death and may be assessed by a medical score (e.g. Acute Physiology And Chronic Health Evaluation (APACHE, APACHE II)).
8. A method according to embodiments 1 to 6, wherein the worsening organ function comprises worsening of renal function (WRF), worsening of cardiovascular function, worsening of hepatic function and worsening of respiratory function and may be assessed by an increasing sequential organ failure assessment (SOFA) score, multiple organ dysfunction score (MODS) or simplified acute physiology score (SAPS, SAPS II).
9. A method according to any of the preceding embodiments, wherein said subject suffers from a disease or condition, e.g. a chronic or acute disease or acute condition.
10. A method according to any of the preceding embodiments, wherein the disease the subject is suffering from may be selected from the group comprising severe infections as e.g. meningitis, Systemic inflammatory Response-Syndrome (SIRS), sepsis; other diseases as diabetes, cancer, acute and chronic vascular diseases as e.g. heart failure, myocardial infarction, stroke, atherosclerosis; shock as e.g. septic shock and organ dysfunction as e.g. kidney dysfunction, liver dysfunction, burnings, surgery, traumata.
11. A method according to any of the preceding embodiments, where said therapeutic measures are selected from the group comprising fluid resuscitation, vasopressors/inotropes, renal replacement therapy, antibiotics, hydrocortisone, insulin, enteral nutrition/parenteral nutrition.
12. A method according to any of the preceding embodiments, wherein the level of said fragment of pre-pro-Adrenomedullin selected from the group comprising MR-proADM, CT-proADM and/or PAMP of at least 5 amino acids is determined by an immunoassay using at least one binder selected from the group comprising a binder to MR-proADM or a fragment thereof and/or to CT-proADM or a fragment thereof and/or to PAMP or a fragment thereof, respectively.
13. A method according to any of the preceding embodiments, wherein for the correlation an elevated level of said fragment of pre-pro-Adrenomedullin selected from the group comprising MR-proADM, CT-proADM and/or PAMP or fragments thereof above a certain threshold is predictive for an enhanced risk for an adverse outcome, and/or a level of said fragment of pre-pro-Adrenomedullin or fragments thereof below a certain threshold is predictive for a reduced risk for an adverse outcome.
14. A method according to any of the preceding embodiments, wherein said threshold is an upper concentration determined for a healthy reference population, such as the 90th, 95th or 99th percentile.
15. A method according to any of the preceding embodiments, wherein for the correlation of the level of the fragment of pre-pro-Adrenomedullin the determination of the level of said fragment is performed at least twice and wherein a decrease of the second measured level of said fragment in comparison to the measured first level of said fragment, is predictive for a reduced risk for an adverse outcome.
16. A method according to any of the preceding embodiments, wherein for the correlation of the level of the fragment of pre-pro-Adrenomedullin the determination of the level of said fragments is performed at least twice and wherein an increase of the second measured level of said fragment in comparison to the measured first level of said fragment, is predictive for an enhanced risk for an adverse outcome.
17. A method according to any of the preceding embodiments, wherein an immunoassay is used for determining the level of MR-proADM and/or CT-proADM and/or PAMP, and wherein the assay sensitivity is <0.5 nmol/L, preferably <0.4 nmol/L and more preferably <0.2 nmol/L for MR-pro ADM and/or <100 pmol/L, preferably <75 pmol/L and more preferably <50 pmol/L for CT-proADM, and/or <0.3 pmol/L, preferably <0.2 pmol/L and more preferably <0.1 pmol/L for PAMP.
18. A method according to any of the preceding embodiments, wherein said bodily fluid may be selected from the group comprising blood, serum, plasma, urine, cerebrospinal fluid (CSF), and saliva.
19. A method according to any of the preceding embodiments, wherein additionally at least one clinical parameter is determined and additionally considered in the correlations selected from the group comprising age, gender, SOFA score (or subscores thereof), SAPSII score, BUN, Sodium, Potassium, Creatinine, Bilirubin, Platelets count, arterial pH, Hematocrit, White Blood count, HCO³⁻, invasive mechanical ventilation/non-invasive mechanical ventilation, hemodynamic characteristics (incl. Blood pressure, systolic and diastolic, Mean arterial pressure, Central venous pressure, Heart rate), Fluid balance, Urine output, Base excess, Chloride, CRP, PCT, BNP or NT-proBNP, Troponin T or Troponin I, Pro-Enkephalin, Hemoglobin, Glucose, Lactate, INR, Alkaline phosphatase, AST, ALT, Gamma GT, Total protein, Albumin, Temperature, Respiratory rate, PaO₂ and FiO₂, therapeutic measures (fluid resuscitation, vasopressors/inotropes, Renal replacement therapy, antibiotics, Hydrocortisone, Insulin, Enteral nutrition/Parenteral nutrition, pre-existing co-morbidities, chronic medication.
20. A method according to any of the preceding embodiments in order to stratify said subjects into risk groups.

[symbol]

### Example 1

Generation of Antibodies and Determination of their Affinity Constants

Several human and murine antibodies were produced and their affinity constants were determined (see Table 2).

Peptides/Conjugates for Immunization:

Peptides for immunization were synthesized, see Table 2, (JPT Technologies, Berlin, Germany) with an additional N-terminal Cystein (if no Cystein is present within the selected ADM-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio.

The murine antibodies were generated according to the following method:

A Balb/c mouse was immunized with 100 μg Peptide-BSA-Conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intra-venous injection.

Splenocytes from the immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium [RPMI 1640 culture medium supplemented with 20% fetal calf serum and HAT-Supplement]. After two weeks the HAT medium is replaced with HT Medium for three passages followed by returning to the normal cell culture medium.

The cell culture supernatants were primary screened for antigen specific IgG antibodies three weeks after fusion. The positive tested microcultures were transferred into 24-well plates for propagation. After retesting, the selected cultures were cloned and recloned using the limiting-dilution technique and the isotypes were determined (see also Lane, R. D. 1985. J. Immunol. Meth. 81: 223-228; Ziegler et al. 1996. Horm. Metab. Res. 28: 11-15).

Mouse Monoclonal Antibody Production:

Antibodies were produced via standard antibody production methods (Marx et al, 1997. Monoclonal Antibody Production, ATLA 25, 121) and purified via Protein A. The antibody purities were >95% based on SDS gel electrophoresis analysis.

Human Antibodies:

Human Antibodies were produced by means of phage display according to the following procedure:

The human naive antibody gene libraries HALT/8 were used for the isolation of recombinant single chain F-Variable domains (scFv) against adrenomedullin peptide. The antibody gene libraries were screened with a panning strategy comprising the use of peptides containing a biotin tag linked via two different spacers to the adrenomedullin peptide sequence. A mix of panning rounds using non-specifically bound antigen and streptavidin bound antigen were used to minimize background of non-specific binders. The eluted phages from the third round of panning have been used for the generation of monoclonal scFv expressing E. coli strains. Supernatant from the cultivation of these clonal strains has been directly used for an antigen ELISA testing (see also Hust et al. 2011. Journal of Biotechnology 152, 159-170; Schütte et al. 2009. PLoS One 4, e6625).

Positive clones have been selected based on positive ELISA signal for antigen and negative for streptavidin coated micro titer plates. For further characterizations the scFv open reading frame has been cloned into the expression plasmid pOPE107 (Hust et al., J. Biotechn. 2011), captured from the culture supernatant via immobilised metal ion affinity chromatography and purified by a size exclusion chromatography.

Affinity Constants:

To determine the affinity of the antibodies to Adrenomedullin, the kinetics of binding of Adrenomedullin to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare). (Lorenz et al. 2011. Antimicrob Agents Chemother. 55(1): 165-173).

The monoclonal antibodies were raised against the below depicted ADM regions of human and murine ADM, respectively. The following table represents a selection of obtained antibodies used in further experiments. Selection was based on target region:

TABLE 2

| | | | | Affinity |
|---|---|---|---|---|
| SEQ ID | Antigen/ | ADM | Desig- | constants |
| NO: | Immunogen | Region | nation | Kd (M) |
| SEQ ID: | YRQSMNNFQ | 1-21 | NT-H | 5.9 × 10⁻⁹ |
| 19 | GLRSFGCRF | | | |
| | GTC | | | |
| SEQ ID: | CTVQKLAHQ | 21-32 | MR-H | 2 × 10⁻⁹ |
| 24 | IYQ | | | |
| SEQ ID: | CAPRSKISP | C-42- | CT-H | 1.1 × 10⁻⁹ |
| 20 | QGY-NH₂ | 52 | | |
| SEQ ID: | YRQSMNQGS | 1-19 | NT-M | 3.9 × 10⁻⁹ |
| 25 | RSNGCRFGT | | | |
| C | | | | |

Generation of Antibody Fragments by Enzymatic Digestion:

The generation of Fab and F(ab)₂ fragments was done by enzymatic digestion of the murine full length antibody NT-M. Antibody NT-M was digested using a) the pepsin-based F(ab)₂ Preparation Kit (Pierce 44988) and b) the papain-based Fab Preparation Kit (Pierce 44985). The fragmentation procedures were performed according to the instructions provided by the supplier. Digestion was carried out in case of F(ab)₂-fragmentation for 8 h at 37° C. The Fab-fragmentation digestion was carried out for 16 h, respectively.

Procedure for Fab Generation and Purification:

The immobilized papain was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample where added to the spin column tube containing the equilibrated Immobilized Papain. Incubation time of the digestion reaction was done for 16 h on a tabletop rocker at 37° C. The column was centrifuged at 5000×g for 1 minute to separate digest from the Immobilized Papain. Afterwards the resin was washed with 0.5 ml PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 ml of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Coulter and Harris 1983. J. Immunol. Meth. 59, 199-203; Lindner et al. 2010. Cancer Res. 70, 277-87; Kaufmann et al. 2010. PNAS. 107, 18950-5; Chen et al. 2010. PNAS. 107, 14727-32; Uysal et al. 2009 J. Exp. Med. 206, 449-62; Thomas et al. 2009. J. Exp. Med. 206, 1913-27; Kong et al. 2009 J. Cell Biol. 185, 1275-840).

Procedure for Generation and Purification of F(ab′)₂ Fragments:

The immobilized Pepsin was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample where added to the spin column tube containing the equilibrated Immobilized Pepsin. Incubation time of the digestion reaction was done for 16 h on a tabletop rocker at 37° C. The column was centrifuged at 5000×g for 1 minute to separate digest from the Immobilized Papain. Afterwards the resin was washed with 0.5 mL PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 mL of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Mariani et al. 1991. Mol. Immunol. 28: 69-77; Beale 1987. Exp Comp Immunol 11:287-96; Ellerson et al. 1972. FEBS Letters 24(3):318-22; Kerbel and Elliot 1983. Meth Enzymol 93:113-147; Kulkarni et al. 1985. Cancer Immunol Immunotherapy 19:211-4; Lamoyi 1986. Meth Enzymol 121:652-663; Parham et al. 1982. J Immunol Meth 53:133-73; Raychaudhuri et al. 1985. Mol Immunol 22(9):1009-19; Rousseaux et al. 1980. Mol Immunol 17:469-82; Rousseaux et al. 1983. J Immunol Meth 64:141-6; Wilson et al. 1991. J Immunol Meth 138:111-9).

NT-H-Antibody Fragment Humanization:

The antibody fragment was humanized by the CDR-grafting method (Jones et al. 1986. Nature 321, 522-525).

The following steps where done to achieve the humanized sequence:

Total RNA extraction: Total RNA was extracted from NT-H hybridomas using the Qiagen kit.

First-round RT-PCR: QIAGEN® OneStep RT-PCR Kit (Cat No. 210210) was used. RT-PCR was performed with primer sets specific for the heavy and light chains. For each RNA sample, 12 individual heavy chain and 11 light chain RT-PCR reactions were set up using degenerate forward primer mixtures covering the leader sequences of variable regions. Reverse primers are located in the constant regions of heavy and light chains. No restriction sites were engineered into the primers.

Reaction Setup: 5× QIAGEN® OneStep RT-PCR Buffer 5.0 μl, dNTP Mix (containing 10 mM of each dNTP) 0.8 μl, Primer set 0.5 μl, QIAGEN® OneStep RT-PCR Enzyme Mix 0.8 μl, Template RNA 2.0 μl, RNase-free water to 20.0 μl, Total volume 20.0 μl PCR condition: Reverse transcription: 50° C., 30 min; Initial PCR activation: 95° C., 15 min Cycling: 20 cycles of 94° C., 25 sec; 54° C., 30 sec; 72° C., 30 sec; Final extension: 72° C., 10 min Second-round semi-nested PCR: The RT-PCR products from the first-round reactions were further amplified in the second-round PCR. 12 individual heavy chain and 11 light chain RT-PCR reactions were set up using semi-nested primer sets specific for antibody variable regions.

Reaction Setup: 2× PCR mix 10 μl; Primer set 2 μl; First-round PCR product 8 μl; Total volume 20 μl; Hybridoma Antibody Cloning Report PCR condition: Initial denaturing of 5 min at 95° C.; 25 cycles of 95° C. for 25 sec, 57° C. for 30 sec, 68° C. for 30 sec; Final extension is 10 min 68° C.

After PCR is finished, run PCR reaction samples onto agarose gel to visualize DNA fragments amplified. After sequencing more than 15 cloned DNA fragments amplified by nested RT-PCR, several mouse antibody heavy and light chains have been cloned and appear correct. Protein sequence alignment and CDR analysis identifies one heavy chain and one light chain. After alignment with homologous human framework sequences the resulting humanized sequence for the variable heavy chain is the following: see FIG. 5. As the amino acids on positions 26, 40 and 55 in the variable heavy chain and amino acid on position 40 in the variable light are critical to the binding properties, they may be reverted to the murine original. The resulting candidates are depicted below. (Padlan 1991. Mol. Immunol. 28, 489-498; Harris and Bajorath. 1995. Protein Sci. 4, 306-310).

Annotation for the antibody fragment sequences (SEQ ID No.: 11-18 and 26-27): bold and underline are the CDR 1, 2, 3 chronologically arranged; italic are constant regions; hinge regions are highlighted with bold letters and the histidine tag with bold and italic letters.

SEQ ID No. 11 (AM-VH-C):
QVQLQQSGAELMKPGASVKISCKAT [symbol] IEWVKQRPGHGLEWIGE [symbol] [symbol]
NYNEKFKGKATITADTSSNTAYMQLSSLTSEDSAVYYC [symbol] WGQGTTLT
VSSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPK [symbol]
SEQ ID No. 12 (AM-VH1):
QVQLVQSGAEVKKPGSSVKVSCKAS [symbol] ISWVRQAPGQGLEWMGR [symbol] [symbol]
NYAQKFQGRVTITADESTSTAYMELSSLRSEDTAVYYC [symbol] WGQGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK [symbol]
SEQ ID No. 13 (AM-VH2-E40):
QVQLVQSGAEVKKPGSSVKVSCKAS [symbol] IEWVRQAPGQGLEWMGR [symbol] [symbol]
NYAQKFQGRVTITADESTSTAYMELSSLRSEDTAVYYC [symbol] WGQGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK [symbol]
SEQ ID No. 14 (AM-VH3-T26-E55):
QVQLVQSGAEVKKPGSSVKVSCKAT [symbol] ISWVRQAPGQGLEWMGE [symbol] [symbol]
NYAQKFQGRVTITADESTSTAYMELSSLRSEDTAVYYC [symbol] WGQGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK [symbol]
SEQ ID No. 15 (AM-VH4-T26-E40-E55):
QVQLVQSGAEVKKPGSSVKVSCKAT [symbol] IEWVRQAPGQGLEWMGE [symbol] [symbol]
NYAQKFQGRVTITADESTSTAYMELSSLRSEDTAVYYC [symbol] WGQGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK [symbol]
SEQ ID No. 16 (AM-VL-C):
DVLLSQTPLSLPVSLGDQATISCRSS [symbol] LEWYLQKPGQSPKLLIY [symbol] N
RFSGVPDRFSGSGSGTDFTLKISRVEAEDLGVYYC [symbol] FGGGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID No. 17 (AM-VL1):
DVVMTQSPLSLPVTLGQPASISCRSS [symbol] LNWFQQRPGQSPRRLIY [symbol] N
RDSGVPDRFSGSGSGTDFTLKISRVEAEDVGVYYC [symbol] FGQGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID No. 18 (AM-VL2-E40):
DVVMTQSPLSLPVTLGQPASISCRSS [symbol] LEWFQQRPGQSPRRLIY [symbol] N
RDSGVPDRFSGSGSGTDFTLKISRVEAEDVGVYYC [symbol] FGQGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID No. 26 (heavy chain of HAM8101):
QVQLVQSGAEVKKPGSSVKVSCKAS [symbol] IEWVRQAPGQGLEWIGE [symbol] [symbol]
NYNQKFQGRVTITADTSTSTAYMELSSLRSEDTAVYYC [symbol] WGQGT
TVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID No. 27 (light chain of HAM8101):
DVVLTQSPLSLPVTLGQPASISCRSS [symbol] LEWYLQRPGQSPRLLIY [symbol] NR
FSGVPDRFSGSGSGTDFTLKISRVEAEDVGVYYC [symbol] FGGGTKLEIKRTVAA
PSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC

### Example 2

The influence of anti-NT-H Adrenomedullin antibody HAM8101 (Adrecizumab) has been explored on clinical and laboratory parameters of septic pigs in a two-hit model. First hit was a hemorrhagic shock, and second hit was sepsis induction by application of an E. coli fibrin clot (peritoneal contamination and infection; PCI). HAM8101 was administered at the time point, when sepsis was induced.

Materials and Methods

Animal Strain: Sus scrofa domestica (Deutsche Landrasse) 14-16 weeks, 30-35 kg

Group size: 6

Groups:

a) PCI+vehicle

b) PCI+HAM8101

Test Materials

HAM8101 (Adrecizumab) Lot No.: HAM-160714-FiB in 20 mM His/HCl pH 6.0

Vehicle: 20 mM His/HCl pH 6.0

Study Execution

Animals

We anaesthetised and ventilated 16 female German Landrace pigs (n=16; mean±standard deviation (SD) 33±1.5 kg body weight (BW)) and followed the standard procedures for laboratory animal care. This study was approved by the institutional and local committee on the care and use of animals (Landesamt für Natur, Umwelt and Verbraucherschutz Nordrhein-Westfalen, Germany, 84-02.04.2015.A037).

General Anesthesia and Catheterisation

Animals were premedicated with azaperone (1-2 mg/kg BW) and ketamine (10 mg/kg BW), and general anesthesia was induced by intravenous injection of propofol (1-2 mg/kg BW). The animals were orally intubated and placed in the supine position. General anaesthesia was maintained with infusions of propofol and fentanyl. Controlled pressure mode ventilation was chosen to ventilate the animals with an inspiratory oxygen fraction of 0.5, an inspiratory/expiratory ratio of 1:1.5, PEEP set to 5 cm H₂O and a tidal volume of 8-10 ml/kg BW. The respiratory rate was set to maintain a PaCO₂ of 3.5-4.5 kPa. The body core temperature was maintained above 37.5° C. with a warming blanket. Two central venous catheter were inserted into the external jugular vein and the femoral vein and an arterial PICCO catheter was inserted into the femoral artery by transcutaneous puncture.

At the end of the study the animals were euthanised in the presence of a veterinarian with a lethal dose of Narcoren® (Merial, Hallbergmoos, Germany) while they were still under deep narcosis.

Escherichia coli Fibrin Clot

In this model, we used an E. coli-fibrin clot with 7-9×10¹¹ colony-forming units (CFUs) per kg/BW to induce septic shock.

Haemodynamic Measurements

All intravascular pressure measurements were referenced to the mid-chest level, and values were obtained at end-expiration. Heart rate, mean arterial pressure (MAP), central venous pressure (CVP) and stroke volume variation (SVV) were recorded continuously. Cardiac output was measured using transpulmonary thermodilution (PICCO, Pulsion medical systems, Feldkirchen, Germany). Extravascular lung water (EVLW), intrathoracic blood volume (ITB V) and global end-diastolic volume (GEDV) were calculated using standard formula.

Laboratory

Blood gas analyses were conducted using a standard blood gas oximetry system (ABL 800; Radiometer, Copenhagen, Denmark) with a co-oximeter. Blood count, electrolytes, creatinine, urea and liver enzymes were determined using standard laboratory techniques. We measured neutrophil gelatinase-associated lipocalin (NGAL) by an enzyme-linked immunosorbent assay using a commercially available kit (Pig NGAL ELISA Kit, BioPorto, Hellerup, Denmark). Creatinine clearance was measured as an estimate of the glomerular filtration rate (ClCrea=Ucrea×Uvol/Pcrea×duration of urine collection period; Ucrea=urine creatinine concentration; Uvol=urine volume during the collection period; Pcrea=serum creatinine concentration). We measured the BW of the animals before and after the experiment. Wet/dry ratio of the lung was determined by weighing a part of the left upper lobe immediately after the experiment and after 5 days. Blood coagulation was analysed performing a rotational thromboelastometry (ROTEM delta, TEM international, Basel, Switzerland). Levels of cytokines (IL-6 and TNF-α) in plasma were assessed by an enzyme-linked immunosorbent assay using commercially available kits specific for pigs.

Bioactive adrenomedullin (bio-ADM) was measured using a novel chemiluminescence immunoassay provided by Sphingotec GmbH (Hennigsdorf, Germany), as previously described (Marino et al. 2014. Crit Care 18:R34). In brief, in a one-step sandwich chemiluminescence immunoassay, based on Acridinium NHS-ester labeling for the detection of bioactive ADM in unprocessed, neat plasma, it uses two mouse monoclonal antibodies, one directed against the midregion (solid phase), and the other directed against the amidated C-terminal moiety of ADM (labelled antibody). The assay utilizes 50 μL of plasma samples/calibrators and 200 μL of labelled detection antibody. The analytical assay sensitivity is 2 pg/mL. The assay is suitable for measuring bio-ADM from numerous mammalian species, including humans and pigs, and it detects both free bio-ADM and bio-ADM, when HAM 8101 (Adrecizumab) is bound to it (Weber et al. 2017. J Appl Lab Medicine, in press).

Plasma MR-proADM was measured with the B⋅R⋅A⋅H⋅M⋅S MR-proADM KRYPTOR assay according to the manufacturer's instructions (Thermo Fisher, Hennigsdorf, Germany).

Experimental Protocol

During catheterisation animals received 10 ml/kg BW/hr of a balanced crystalloid solution. Haemorrhagic shock was induced by bleeding the animals via the femoral vein catheter. The animals were bled until half of the baseline MAP was reached. Haemorrhagic shock was maintained for 45 minutes, followed by fluid resuscitation with a balanced crystalloid solution in order to restore baseline mean arterial pressure. 2 hours after haemorrhagic shock the blood collected during haemorrhagic shock was re-transfused. As second hit, sepsis was induced using an E. coli-laden clot placed into the abdominal cavity 6 hours after haemorrhagic shock. Animals were randomly allocated to receive either the adrenomedullin antibody or the vehicle solution. The solutions were delivered in neutral bags and the groups were blinded for the investigators and marked with A or B. The therapy with the antibody or vehicle solution started immediately after the induction of sepsis. 2 mg/kg BW of the antibody/vehicle solution were infused over a period of 30 minutes. 4 hours after sepsis had been induced the therapy of the septic shock started using balanced crystalloids and noradrenalin on demand Volume replacement and vasopressor were titrated to maintain a central venous pressure of 8-12 mmHg, a mean arterial pressure above 65 mmHg and a central venous oxygen saturation of 70%, as recommended by the Surviving Sepsis Campaign. Sepsis therapy continued for another 8 hours. Measurements were performed before haemorrhagic shock, before sepsis induction and 1, 2, 3, 4, 6, 8, 10 and 12 hours after sepsis induction. Haemorrhagic and septic shock were not performed on the animals of the SHAM-groups, but apart from that, they received the same treatment including all intravascular catheter, the median laparotomy and the blinded application of the antibody/vehicle solution.

Results:

As expected, induction of sepsis lead to an increase of plasma bio-ADM in the vehicle animals. Surprisingly, upon administration of HAM 8101, the apparent plasma bio-ADM concentration rose considerably quicker and to a higher level than in the vehicle animals (FIG. 2A). Considering the vast molar excess of HAM 8101 over the endogenous bio-ADM it has to be assumed that the measured plasma ADM concentration after administration of HAM 8101 in fact mainly represents bio-ADM complexed with the HAM 8101 antibody. In order to investigate, whether the observed HAM8101-induced faster and more pronounced increase of plasma bio-ADM was due to enhanced expression of the ADM gene and/or release of ADM gene-products, plasma concentrations of another peptide derived from the ADM precursor peptide were measured, namely MR-proADM (mid-regional pro-Adrenomedullin). As shown in FIG. 2B, plasma levels of MR-proADM increased similarly upon sepsis induction independent from whether animals were treated with HAM8101 or vehicle. Thus, it appears that the HAM8101-induced faster and more pronounced increase of plasma ADM is not due to enhanced expression of the ADM gene and/or release of ADM gene-products.

The overproportional increase of plasma bio-ADM observed after administration of HAM 8101 in comparison to vehicle animals was not associated with a worse outcome:

Animals having received HAM 8101 required less volume resuscitation to achieve a target mean arterial pressure than the vehicle animals (FIG. 3).

Only one third of the animals having received HAM 8101 required noradrenaline administration on top of volume resuscitation to achieve a target mean arterial pressure, whereas all the vehicle animals required noradrenaline (FIG. 4).

As shown in FIG. 4, among the HAM 8101-treated animals, one third required noradrenaline, whereas two thirds did not. These two groups differed in their development of plasma MR-proADM concentrations (FIG. 5). Those animals developing shock, e.g. requiring noradrenaline on top of fluid resuscitation, had considerably higher MR-proADM concentrations than the other animals. The latter, successfully treated animals had lower MR-proADM concentrations than the vehicle group.

Taken together, the data demonstrate that—surprisingly—under treatment with HAM 8101, measurement of bio-ADM is not suitable to monitor the subject's risk for adverse outcome. In contrast, measurement of MR-proADM, as an example of another fragment derived from the ADM-precursor peptide to which HAM 8101 does not bind, is suitable for monitoring the stimulation or down-regulation of the ADM system in such condition, as it is not immediately influenced by administration of HAM 8101 and over time correlates with clinical outcome.

### Example 3

The influence of a monoclonal anti-C-terminal Adrenomedullin antibody generated against SEQ ID No.: 20 (HAM2302) on ADM levels in Male Wistar rats (Charles River, Sulzfeld) has been explored.

Sampling of blood (K₃-EDTA-Plasma≤150 μL or 3-4 mL at terminal bleeding (−80° C.)) was done at time points −3 d, 12 min, 1 h, 3 h, 6 h, 24 h, 48, 4 d, 7 d, 10 d. The animals were treated with a single intravenous injection (5 mL/kg bodyweight).

Injection and sampling was successful for all animals. Antibody-treated animals showed no obvious signs of toxic effects.

| | | |
|---|---|---|
| Treatment | Dose | No. of animals |

| | | | |
|---|---|---|---|
| HAM 2302 | 20 | mg/kg | 6 |
| PBS | 0 | mg/kg | 6 |

Measurement of ADM and Free HAM2302 in Plasma

In order to measure, Adrenomedullin in plasma samples containing the HAM2302 the tADM-Assay was utilized. This assay uses an N-terminal anti-ADM antibody generated against SEQ ID No.: 25 as solid phase (HAM 1112) and a mid-regional anti-ADM antibody generated against amino acid 27 to 39 of ADM (AHQIYQFTDK DKD; SEQ ID No.: 22) (HAM 2903) as tracer. Calibrators made of synthetic rat bio-ADM (1-50)-NH₂, (YRQSMNQGSRSTGCRFGTCTMQKLAHQIYQFTDKDKDGMAPRNKISPQGY-NH₂, Seq ID No.: 23) are used to quantify the ADM in the sample. In contrast to the bio-ADM assay mentioned above, the tADM-assay does not specifically measure the amidated form of ADM, but can detect all forms of ADM.

Purified monoclonal HAM 2903 antibody (1 g/L) was labeled by incubation in 10% labeling buffer (500 mmol/L sodium phosphate, pH 8.0) with 1:5 mol/L ratio of MACN-acridinium-NHS-ester (1 g/L, InVent GmbH) for 30 min at 22° C. in the dark. After adding 5% 1 mol/L Tris-HCl, pH 8.0, for 10 min, the HAM 2903 antibody was separated from free label via CentriPure P5 columns (emp Biotech GmbH) and by size-exclusion HPLC on Protein KW-803 (Shodex, Showa Denko Europe).

White polystyrene microtiter plates (Greiner Bio-One International AG) were coated (18 h at 22° C.) with monoclonal HAM 1112 antibody (1.5 μg/0.2 mL per well 50 mmol/L Tris-HCl, 100 mmol/L NaCl, pH 7.8). After wash and blocking with 30 g/L Karion, 5 g/L bovine serum albumin (protease free), 6.5 mmol/L monopotassium phosphate, 3.5 mmol/L sodium dihydrogen phosphate (pH 6.5) for 1.5 hours, the plates were vacuum-dried.

Synthetic rat ADM (rADM) (peptides & elephants) was serially diluted using 20 mmol/L potassium hydrogenphosphate, 0.5 g/L bovine serum albumin (BSA), 6 mmol/L sodium EDTA, 50 μmon amastatin, 100 μmon leupeptin; pH 8.

Fifty μL of samples/calibrators were pipetted into coated microtiter plates. After addition of 150 μL of labeled C-terminal antibody HAM 2302, microtiter plates were incubated for 20 h at 2-8° C. under agitation. Unbound tracer was removed by washing five times (each 350 μL per well) with washing solution (400 mmol/L Tris, 1 g/L Tween 20, 3 mol/L NaCL, pH 7.5). Wellbound chemiluminescence was measured for 1 s per well by using the Centro LB 960 microtiter plate luminescence reader (Berthold Technologies).

For determination of free HAM2302, the Adrenomab-1 Assay was used. A mid-regional anti-ADM antibody generated against amino acid 21-32 (SEQ ID No.: 24) functions as solid phase.

The MACN-labelled C-terminal Ab (HAM 2302)) functions as tracer. Quantification of the HAM 2302 in a sample utilizes a competitive assay design. Calibrators made of non-labeled HAM 2302 together with a constant hADM concentration (10 ng/mL) are utilized to generate the standard curve to determine the concentration in unknown samples. With increasing concentrations of the calibrator/antibody in the sample, the measured light signal declines because less tracer can bind to adrenomedullin.

Purified monoclonal HAM 2302 (1 g/L) was labeled by incubation in 10% labeling buffer (500 mmol/L sodium phosphate, pH 8.0) with 1:4.5 mol/L ratio of MACN-acridinium-NHS-ester (1 g/L, InVent GmbH) for 30 min at 22° C. in the dark. After adding 5% 1 mol/L Tris-HCl, pH 8.0, for 10 min, HAM 2302 was separated from free label via CentriPure P10 columns (emp Biotech GmbH) and by size-exclusion HPLC on Protein KW-803 (Shodex, Showa Denko Europe).

White polystyrene microtiter plates (Greiner Bio-One International AG) were coated (18 h at 20° C.) with monoclonal midregional antibody against amino acid 21-32 of ADM (1 μg/0.2 mL per well 50 mmol/L Tris-HCl, 100 mmol/L NaCl, pH 7.8). After blocking with 30 g/L Karion, 5 g/L BSA (protease free), 6.5 mmol/L monopotassium phosphate, 3.5 mmol/L sodium dihydrogen phosphate (pH 6.5) for 1.5 hours, the plates were vacuum-dried.

A serial dilution of HAM 2302 prepared with phosphate-buffered saline (PBS), 2.5 g/L bovine serum albumin pH 7.4 was used.

Fifty μL of samples/calibrators and 100 μL of a 10 ng/mL hADM in phosphate-buffered saline (PBS), 2.5 g/L bovine serum albumin pH 7.4 were pipetted into coated microtiter plates. After one-hour incubation at 2-8° C. under agitation, 100 μL of labeled HAM 2302, were pipetted into coated microtiter plates. After incubation of the plates for another 2.5 hours at 2-8° C. under agitation, unbound tracer was removed by washing five times (each 350 μL per well) with washing solution (20 mmol/L PBS, 1 g/L Triton X-100, pH 7.4). Wellbound chemiluminescence was measured for 1 s per well by using the Centro LB 960 microtiter plate luminescence reader (Berthold Technologies).

Results:

In samples of HAM2302 treated animals plasma concentrations free HAM2302 measured at 3 h post-injection were 481.9 μg/mL (±46.8 μg/mL). Mean ADM concentration in vehicle animals was 14.6±3.75 pg/ml. The concentration of Adrenomedullin in HAM2302-treated animals was about 100-fold higher (1411±67 pg/mL) compared to vehicle animals (FIG. 6).

Sequence Listing
SEQ ID NO. 1
(mature Adrenomedullin (mature ADM);
amidated ADM; bio-ADM): amino
acids 95-146 CONH₂ of preproADM
YRQSMN NFQGLRSFGC RFGTCTVQKL
AHQIYQFTDK DKDNVAPRSK ISPQGY-
CONH₂
SEQ ID NO. 2
(pre-pro-Adrenomedullin (pre-proADM)):
amino acids 1-185
MKLVSVALMYLGSLAFLGADTARLDVASEFRKKW
NKWALSRGKRELRMSSSYPTGLADVKAGPAQTLI
RPQDMKGASRSPEDSSPDAARIRVKRYRQSMNNF
QGLRSFGCRFGTCTVQKLAHQIYQFTDKDKDNVA
PRSKISPQGYGRRRRRSLPEAGPGRTLVSSKPQA
HGAPAPPSGSAPHFL
SEQ ID NO. 3
(Proadrenomedullin N-20 terminal peptide,
PAMP): amino acids 22-41 of preproADM
ARLDVASEF RKKWNKWALS R
SEQ ID NO. 4
(Midregional proAdrenomedullin, MR-proADM):
amino acids 45-92 of preproADM
ELRMSS SYPTGLADVK AGPAQTLIRP
QDMKGASRSP EDSSPDAARI RV
SEQ ID NO. 5
(C-terminal proAdrenomedullin,
CT-proADM): amino acids 148-185 of
preproADM
RRR RRSLPEAGPG RTLVSSKPQA
HGAPAPPSGS APHFL
SEQ ID NO. 6:
GYTFSRYW
SEQ ID NO. 7:
ILPGSGST
SEQ ID NO. 8:
TEGYEYDGFDY
SEQ ID NO. 9:
QSIVYSNGNTY
SEQ ID NO. 10:
FQGSHIPYT.
SEQ ID NO. 11 (AM-VH-C):
QVQLQQSGAELMKPGASVKISCKATGYTFSRYWIE
WVKQRPGHGLEWIGEILPGSGSTNYNEKFKGKATT
TADTSSNTAYMQLSSLTSEDSAVYYCTEGYEYDGF
DYWGQGTTLTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPK
SEQ ID NO. 12 (AM-VH1):
QVQLVQSGAEVKKPGSSVKVSCKASGYTFSRYWIS
WVRQAPGQGLEWMGRILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPK
SEQ ID NO. 13 (AM-VH2-E40):
QVQLVQSGAEVKKPGSSVKVSCKASGYTFSRYWIE
WVRQAPGQGLEWMGRILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPK
SEQ ID NO. 14 (AM-VH3-T26-E55):
QVQLVQSGAEVKKPGSSVKVSCKATGYTFSRYWIS
WVRQAPGQGLEWMGEILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPK
SEQ ID NO. 15 (AM-VH4-T26-E40-E55):
QVQLVQSGAEVKKPGSSVKVSCKATGYTFSRYWIE
WVRQAPGQGLEWMGEILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPK
SEQ ID NO. 16 (AM-VL-C):
DVLLSQTPLSLPVSLGDQATISCRSSQSIVYSNGN
TYLEWYLQKPGQSPKLLIYRVSNRFSGVPDRFSGS
GSGTDFTLKISRVEAEDLGVYYCFQGSHIPYTFGG
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC
SEQ ID NO. 17 (AM-VL1):
DVVMTQSPLSLPVTLGQPASISCRSSQSIVYSNGN
TYLNWFQQRPGQSPRRLIYRVSNRDSGVPDRFSGS
GSGTDFTLKISRVEAEDVGVYYCFQGSHIPYTFGQ
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC
SEQ ID NO. 18 (AM-VL2-E40):
DVVMTQSPLSLPVTLGQPASISCRSSQSIVYSNGN
TYLEWFQQRPGQSPRRLIYRVSNRDSGVPDRFSGS
GSGTDFTLKISRVEAEDVGVYYCFQGSHIPYTFGQ
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSP
VTKSFNRGEC
SEQ ID NO. 19 (N-terminal part,
aa 1-21, of adrenomedullin)
YRQSMNNFQGLRSFGCRFGTC
SEQ ID NO. 20 (C-terminal part,
aa 42-52, of adrenomedullin)
APRSKISPQGY-NH₂.
SEQ ID NO. 21 (mid-regional part,
aa 21-42, of adrenomedullin)
CTVQKLAHQIYQFTDKDKDNVA
SEQ ID NO. 22 (mid-regional part
aa 27-39 of adrenomedullin)
AHQIYQFTDK DKD
SEQ ID NO. 23
(rat adrenomedullin amino acid 1-50)
YRQSMNQGSRSTGCRFGTCTMQKLAHQIYQFTDK
DKDGMAPRNKISPQGY-NH₂
SEQ ID NO. 24 (mid-regional part of
adrenomedullin, aa 21-32)
CTVQKLAHQIYQ
SEQ ID NO. 25 (N-terminal part of
murine adrenomedullin, aa 1-19)
YRQSMNQGSRSNGCRFGTC
SEQ ID NO. 28: (Not mentioned in the
sequence listing due to the length
of 3 amino acids)
RVS
SEQ ID NO. 26 (heavy chain of HAM8101)
QVQLVQSGAEVKKPGSSVKVSCKASGYTFSRYWIE
WVRQAPGQGLEWIGEILPGSGSTNYNQKFQGRVTI
TADTSTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKKVEPKSCDKTHTCPPCPAPELLGGPSVFLFP
PKPKDTLMISRTPEVTCVVVDVSHEDPEVKFNWYV
DGVEVHNAKTKPREEQYNSTYRVVSVLTVLHQDWL
NGKEYKCKVSNKALPAPIEKTISKAKGQPREPQVY
TLPPSRDELTKNQVSLTCLVKGFYPSDIAVEWESN
GQPENNYKTTPPVLDSDGSFFLYSKLTVDKSRWQQ
GNVFSCSVMHEALHNHYTQKSLSLSPGK
SEQ ID NO. 27 (light chain of HAM8101)
DVVLTQSPLSLPVTLGQPASISCRSSQSIVYSNGN
TYLEWYLQRPGQSPRLLIYRVSNRFSGVPDRFSGS
GSGTDFTLKISRVEAEDVGVYYCFQGSHIPYTFGG
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC

### FIGURE DESCRIPTION

FIG. 1: Study time schedule in a two-hit pig model

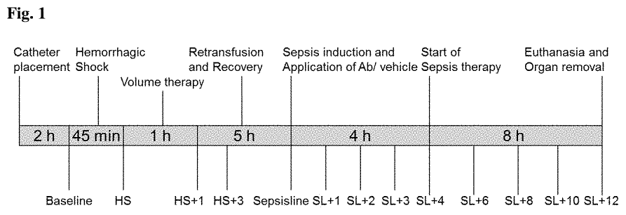

FIG. 2: Plasma bio-ADM (A) and MR-proADM (B): For both groups mean values±SEM are shown. For bio-ADM (A), interaction (7 h-19 h, multivar. Time*Group): 0.003

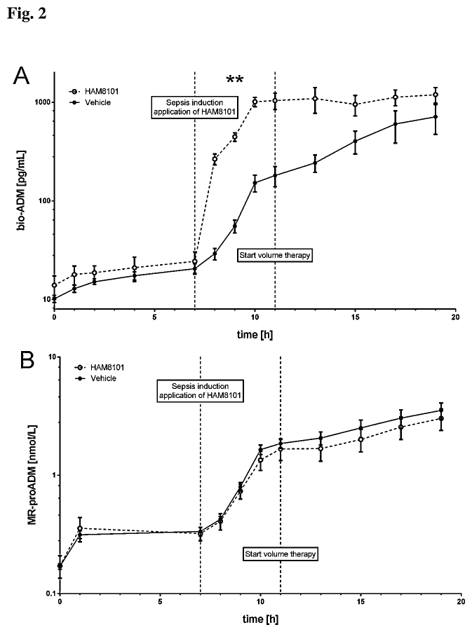

FIG. 3: Fluid resuscitation: For both groups mean values±SEM are shown.

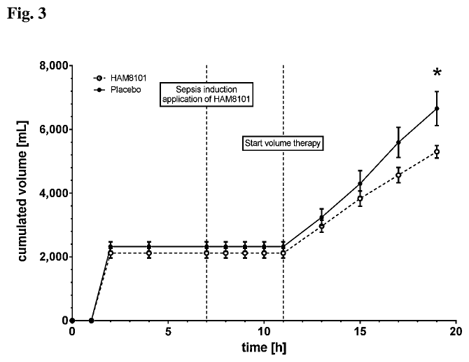

FIG. 4: Frequency of noradrenaline requirement. The percentage of animals requiring noradrenaline on top of fluid resuscitation to achieve the target MAP per group is shown. Chi2 test (19 h):0.014.

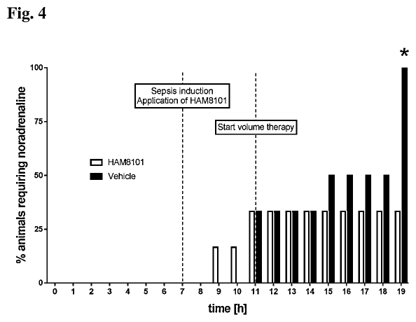

FIG. 5: Plasma MR-proADM depending on therapy success: Shown are mean values±SEM for three groups: HAM 8101-treated and requiring noradrenaline (shock), HAM 8101-treated and not requiring noradrenaline (non-shock), vehicle (all requiring noradrenaline).

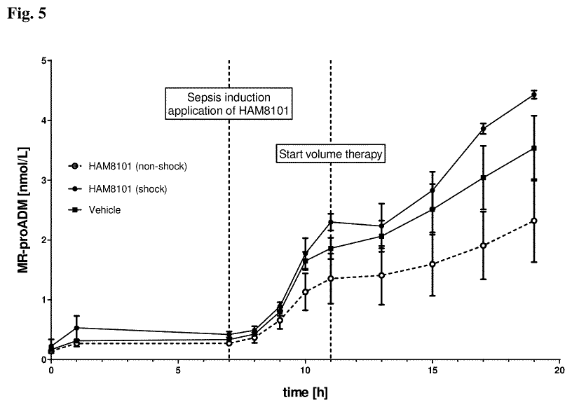

FIG. 6: Comparison of Adrenomedullin concentrations at 3 h post-injection of 20 mg/kg HAM2302 (n=3). Mean Ab concentrations for treated animals are stated below the graph.

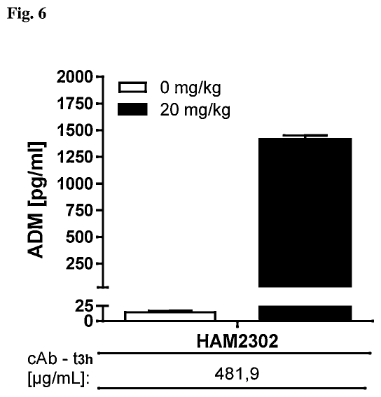

1. A method for monitoring a therapy in a subject, wherein the subject is under treatment with a binder selected from the group comprising an anti-Adrenomedullin (ADM) antibody, anti-body fragment and/or non-Ig Scaffold binding to SEQ ID NO. 1 (amino acid 1-52), comprising
determining the level of a fragment of pre-pro-Adrenomedullin selected from the group comprising Midregional Proadrenomedullin (MR-proADM), C-terminal Proadrenomedullin (CT-proADM) and/or Proadrenomedullin N-terminal 20 peptide (PAMP) or fragments thereof in a bodily fluid obtained from said subject; and
correlating said level of the fragment of pre-pro-Adrenomedullin selected from the group comprising MR-proADM, CT-proADM and/or PAMP with the subject's clinical/medical status of health and/or the risk for an adverse outcome and/or the requirement for adapting therapeutic measures, and
wherein for the determination of the level of said fragments at least one binder binds to a region within the amino acid sequence selected from the group comprising SEQ ID NO. 3, SEQ ID NO. 4 and SEQ ID NO. 5, respectively.

2. A method according to claim 1, wherein the binder for the treatment is an anti-ADM antibody or an anti-adrenomedullin antibody fragment or an anti-ADM non-Ig protein scaffold, wherein said antibody or fragment or scaffold binds to the N-terminal part, aa 1-21, of adrenomedullin:
SEQ ID No. 19
YRQSMNNFQGLRSFGCRFGTC.

3. A method according to claim 1, wherein the binder for the treatment is an anti-ADM antibody or an anti-adrenomedullin antibody fragment or an anti-ADM non-Ig protein scaffold, wherein said antibody or fragment or scaffold binds to the C-terminal part, aa 42-52-amide, of adrenomedullin:
SEQ ID No. 20
APRSKISPQGY-NH₂.

4. A method according to claim 1, wherein the binder for the treatment is an anti-ADM antibody or an anti-adrenomedullin antibody fragment or an anti-ADM non-Ig protein scaffold, wherein said antibody or fragment or scaffold binds to the mid-regional part, aa 21-42, of adrenomedullin:
SEQ ID No. 21
CTVQKLAHQIYQFTDKDKDNVA.

5. A method according to claim 1, wherein the determination of the level of said fragments is performed at least once.

6. A method according to claim 1, wherein the adverse outcome is selected from the group comprising worsening clinical condition such as worsening organ function, and mortality.

7. A method according to claim 1, wherein said subject suffers from a disease or condition, e.g. a chronic or acute disease or acute condition.

8. A method according claim 1, wherein the disease the subject is suffering from may be selected from the group comprising severe infections as e.g. meningitis, Systemic inflammatory Response-Syndrome (SIRS), sepsis; other diseases as diabetes, cancer, acute and chronic vascular diseases as e.g. heart failure, myocardial infarction, stroke, atherosclerosis; shock as e.g. septic shock and organ dysfunction as e.g. kidney dysfunction, liver dysfunction, burnings, surgery, traumata.

9. A method according to claim 1, where said therapeutic measures are selected from the group comprising fluid resuscitation, vasopressors/inotropes, renal replacement therapy, antibiotics, hydrocortisone, insulin, enteral nutrition/parenteral nutrition.

10. A method according to claim 1, wherein the level of said fragment of pre-pro-Adrenomedullin selected from the group comprising MR-proADM, CT-proADM and/or PAMP of at least 5 amino acids is determined by an immunoassay using at least one binder selected from the group comprising a binder to MR-proADM or a fragment thereof and/or to CT-proADM or a fragment thereof and/or to PAMP or a fragment thereof, respectively.

11. A method according to claim 1, wherein for the correlation an elevated level of said fragment of pre-pro-Adrenomedullin selected from the group comprising MR-proADM, CT-proADM and/or PAMP or fragments thereof above a certain threshold is predictive for an enhanced risk for an adverse outcome, and/or a level of said fragment of pre-pro-Adrenomedullin or fragments thereof below a certain threshold is predictive for a reduced risk for an adverse outcome.

12. A method according to claim 1, wherein for the correlation of the level of the fragment of pre-pro-Adrenomedullin the determination of the level of said fragment is performed at least twice and wherein a decrease of the second measured level of said fragment in comparison to the measured first level of said fragment, is predictive for a reduced risk for an adverse outcome.

13. A method according to claim 1, wherein for the correlation of the level of the fragment of pre-pro-Adrenomedullin the determination of the level of said fragments is performed at least twice and wherein an increase of the second measured level of said fragment in comparison to the measured first level of said fragment, is predictive for an enhanced risk for an adverse outcome.

14. A method according to claim 1, wherein said bodily fluid may be selected from the group comprising blood, serum, plasma, urine, cerebrospinal fluid (CSF), and saliva.

15. A method according to claim 1, in order to stratify said subjects into risk groups.


SEQUENCE LISTING (34 sequences)

SEQ ID NO: 1 - protein, 52 aa, Homo sapiens
YRQSMNNFQGLRSFGCRFGTCTVQKLAHQIYQFTDKDKDNVAPRSKISPQGY

SEQ ID NO: 2 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 3 - protein, 20 aa, Homo sapiens
ARLDVASEFRKKWNKWALSR

SEQ ID NO: 4 - protein, 48 aa, Homo sapiens
ELRMSSSYPTGLADVKAGPAQTLIRPQDMKGASRSPEDSSPDAARIRV

SEQ ID NO: 5 - protein, 38 aa, Homo sapiens
RRRRRSLPEAGPGRTLVSSKPQAHGAPAPPSGSAPHFL

SEQ ID NO: 6 - protein, 8 aa, Artificial Sequence
  Description of Artificial Sequence Synthetic peptide
GYTFSRYW

SEQ ID NO: 7 - protein, 8 aa, Artificial Sequence
  Description of Artificial Sequence Synthetic peptide
ILPGSGST

SEQ ID NO: 8 - protein, 11 aa, Artificial Sequence
  Description of Artificial Sequence Synthetic peptide
TEGYEYDGFDY

SEQ ID NO: 9 - protein, 11 aa, Artificial Sequence
  Description of Artificial Sequence Synthetic peptide
QSIVYSNGNTY

SEQ ID NO: 10 - protein, 9 aa, Artificial Sequence
  Description of Artificial Sequence Synthetic peptide
FQGSHIPYT

SEQ ID NO: 11 - protein, 219 aa, Artificial Sequence
  Description of Artificial Sequence Synthetic polypeptide
QVQLQQSGAELMKPGASVKISCKATGYTFSRYWIEWVKQRPGHGLEWIGEILPGSGSTNYNEKFKGKATITADTSSNTAYMQLSSLTSEDSAVYYCTEGYEYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 12 - protein, 219 aa, Artificial Sequence
  Description of Artificial Sequence Synthetic polypeptide
QVQLVQSGAEVKKPGSSVKVSCKASGYTFSRYWISWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 13 - protein, 219 aa, Artificial Sequence
  Description of Artificial Sequence Synthetic polypeptide
QVQLVQSGAEVKKPGSSVKVSCKASGYTFSRYWIEWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 14 - protein, 219 aa, Artificial Sequence
  Description of Artificial Sequence Synthetic polypeptide
QVQLVQSGAEVKKPGSSVKVSCKATGYTFSRYWISWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 15 - protein, 219 aa, Artificial Sequence
  Description of Artificial Sequence Synthetic polypeptide
QVQLVQSGAEVKKPGSSVKVSCKATGYTFSRYWIEWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 16 - protein, 219 aa, Artificial Sequence
  Description of Artificial Sequence Synthetic polypeptide
DVLLSQTPLSLPVSLGDQATISCRSSQSIVYSNGNTYLEWYLQKPGQSPKLLIYRVSNRFSGVPDRFSGSGSGTDFTLKISRVEAEDLGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 219 aa, Artificial Sequence
  Description of Artificial Sequence Synthetic polypeptide
DVVMTQSPLSLPVTLGQPASISCRSSQSIVYSNGNTYLNWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 219 aa, Artificial Sequence
  Description of Artificial Sequence Synthetic polypeptide
DVVMTQSPLSLPVTLGQPASISCRSSQSIVYSNGNTYLEWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 21 aa, Homo sapiens
YRQSMNNFQGLRSFGCRFGTC

SEQ ID NO: 20 - protein, 11 aa, Homo sapiens
APRSKISPQGY

SEQ ID NO: 21 - protein, 22 aa, Homo sapiens
CTVQKLAHQIYQFTDKDKDNVA

SEQ ID NO: 22 - protein, 13 aa, Homo sapiens
AHQIYQFTDKDKD

SEQ ID NO: 23 - protein, 50 aa, Artificial Sequence
  Description of Artificial Sequence Synthetic polypeptide
YRQSMNQGSRSTGCRFGTCTMQKLAHQIYQFTDKDKDGMAPRNKISPQGY

SEQ ID NO: 24 - protein, 12 aa, Homo sapiens
CTVQKLAHQIYQ

SEQ ID NO: 25 - protein, 19 aa, Mus sp.
YRQSMNQGSRSNGCRFGTC

SEQ ID NO: 26 - protein, 448 aa, Artificial Sequence
  Description of Artificial Sequence Synthetic polypeptide
QVQLVQSGAEVKKPGSSVKVSCKASGYTFSRYWIEWVRQAPGQGLEWIGEILPGSGSTNYNQKFQGRVTITADTSTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 219 aa, Artificial Sequence
  Description of Artificial Sequence Synthetic polypeptide
DVVLTQSPLSLPVTLGQPASISCRSSQSIVYSNGNTYLEWYLQRPGQSPRLLIYRVSNRFSGVPDRFSGSGSGTDFTLKISRVEAEDVGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 3 aa, Artificial Sequence
  Description of Artificial Sequence Synthetic peptide
RVS

SEQ ID NO: 29 - protein, 225 aa, Artificial Sequence
  Description of Artificial Sequence Synthetic polypeptide
QVQLQQSGAELMKPGASVKISCKATGYTFSRYWIEWVKQRPGHGLEWIGEILPGSGSTNYNEKFKGKATITADTSSNTAYMQLSSLTSEDSAVYYCTEGYEYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 30 - protein, 225 aa, Artificial Sequence
  Description of Artificial Sequence Synthetic polypeptide
QVQLVQSGAEVKKPGSSVKVSCKASGYTFSRYWISWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 31 - protein, 225 aa, Artificial Sequence
  Description of Artificial Sequence Synthetic polypeptide
QVQLVQSGAEVKKPGSSVKVSCKASGYTFSRYWIEWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 32 - protein, 225 aa, Artificial Sequence
  Description of Artificial Sequence Synthetic polypeptide
QVQLVQSGAEVKKPGSSVKVSCKATGYTFSRYWISWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 33 - protein, 225 aa, Artificial Sequence
  Description of Artificial Sequence Synthetic polypeptide
QVQLVQSGAEVKKPGSSVKVSCKATGYTFSRYWIEWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 34 - protein, 12 aa, Artificial Sequence
  Description of Artificial Sequence Synthetic peptide
CAPRSKISPQGY